(19) United States
(12) Patent Application Publication
(10) Pub. No.: US 2017/0304361 A1
(43) Pub. Date: Oct. 26, 2017

(54) HALOGENATED INSULIN ANALOGUES OF ENHANCED BIOLOGICAL POTENCY

(71) Applicant: CASE WESTERN RESERVE UNIVERSITY, Cleveland, OH (US)

(72) Inventor: Michael A. Weiss, Moreland Hills, OH (US)

(73) Assignee: CASE WESTERN RESERVE UNIVERSITY, Cleveland, OH (US)

(21) Appl. No.: 15/520,710

(22) PCT Filed: Oct. 12, 2015
(86) PCT No.: PCT/US15/55057

§ 371 Date: Apr. 20, 2017

Related U.S. Application Data
(60) Provisional application No. 62/066,187, filed on Oct. 20, 2014.

(51) Int. Cl. A61K 33/30; C07K 14/62; A61K 38/27; A61K 38/28; C07K 14/00; A61K 38/00

(52) U.S. Cl. CPC A61K 33/30; A61K 38/27; A61K 38/28; C07K 14/62; A61K 38/00; C07K 14/00; A61K 2300/00

(57) ABSTRACT

An insulin molecule comprises an Asp substitution at position B10, Glu at one or more of positions corresponding to A8, B28, and B29, and a halogenated phenylalanine at position B24. The analogue may optionally include (i) N-terminal deletion of one, two or three residues from the B chain, (ii) a mono-peptide or dipeptide C-terminal extension of the B-chain containing at least one acidic residue, and (iii) other modifications known in the art to enhance the stability of insulin. Formulations of the above analogues at successive strengths U-100 to U-1000 in soluble solutions at at least pH value in the range 7.0-8.0 in the absence or presence of zinc ions at a molar ratio of 0.00-0.10 zinc ions per insulin analogue monomer. A method of lowering the blood sugar level of a patient comprises administering a physiologically effective amount of the insulin to a patient.

### CROSS REFERENCE TO RELATED APPLICATIONS

This application a national stage application of International Application No. PCT/US2015/055057 filed on Oct. 12, 2015, which claims priority from U.S. Provisional Patent Application No. 62/066,187, filed on Oct. 20, 2014.

### STATEMENT REGARDING FEDERALLY SPONSORED RESEARCH OR DEVELOPMENT

This invention was made with government support under grant numbers DK040949 and DK074176 awarded by the National Institutes of Health. The U.S. government has certain rights in the invention.

### BACKGROUND OF THE INVENTION

This invention relates to polypeptide hormone analogues that exhibit enhanced pharmaceutical properties, such as increased thermodynamic stability and/or enhanced potency. More particularly, this invention relates to insulin analogues that confer rapid action at increased formulation strengths (relative to wild-type insulin) and/or that exhibit increased biological potency per nanomole of the hormone analogue administered to a patient (relative to wild-type insulin). The analogues of the present invention thus consist of two polypeptide chains that contain a novel combination of acidic amino-acid substitutions in the A-chain and/or B chain such that the analogue exhibits (i) enhanced thermodynamic stability in the absence of divalent metal ions, (ii) decreased self-association at protein concentrations greater than or equal to 0.6 mM, and (iii) enhanced biological potency in vivo on a nanomolar basis, i.e., such that, relative to wild-type human insulin, fewer molecules of the insulin analogue are required, on subcutaneous or intravenous injection into a diabetic mammal, to elicit a similar reduction in blood-glucose concentration. To avoid an unfavorable increase mitogenicity, the analogues of the present invention also contain a halogen atom (fluorine, chlorine, bromine, or iodine; F, Cl, Br or I) in the aromatic ring of Phenylalanine at position B24 (such as at the ortho, meta or para position of the aromatic ring) and may optionally contain standard or non-standard amino-acid substitutions at other sites in the A or B domains, such as positions B28 and B29 known in the art to confer rapid action.

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—may have evolved to function optimally within a cellular context but nonetheless may be suboptimal for therapeutic applications. Analogues of such proteins may exhibit improved biophysical, biochemical, or biological properties. A benefit of protein analogues would be to achieve enhanced “on-target” activity (such as metabolic regulation of metabolism leading to reduction in blood-glucose concentration in a diabetic mammal or patient with diabetes mellitus) with decreased unintended and/or unfavorable side effects, such as promotion of the growth of cancer cells. Another benefit of such protein engineering would be preservation of rapid onset of action on concentration of the protein to achieve formulations of higher strength. Yet another example of a societal benefit would be augmented resistance to degradation at or above room temperature, facilitating transport, distribution, and use. An example of a therapeutic protein is provided by insulin. Wild-type human insulin and insulin molecules encoded in the genomes of other mammals bind to insulin receptors is multiple organs and diverse types of cells, irrespective of the receptor isoform generated by alternative modes of RNA splicing or by alternative patterns of post-translational glycosylation. Wild-type insulin also binds with lower but significant affinity to the homologous Type 1 insulin-like growth factor receptor (IGF-1R).

An example of a further medical benefit would be optimization of the pharmacokinetic properties of a soluble insulin analogue formulation such that rapid onset of action (as is characteristic of prandial insulin analogues known in the art at a strength of U-100) is retained in formulations of strengths in the range U-200 through U-1000, i.e., between twofold and tenfold higher than conventional U-100 insulin products (in this nomenclature “U-X” designates X internal units per ml of solution or suspension). Insulin formulations of increased strength promise to be of particular benefit for patients who exhibit marked insulin resistance and may also be of value in internal or external insulin pumps, either to extend the reservoir life or to permit miniaturization of the reservoir in a new generation of pump technologies. Existing insulin products typically exhibit prolonged pharmacokinetic and pharmacodynamics properties on increasing the concentration of the insulin or insulin analogue to achieve formulation strengths>U-200 (200 international units/ml). Such prolongation impairs the efficacy of such products for the prandial control of glycemia on subcutaneous injection and impairs the efficacy and safety of pump-based continuous subcutaneous infusion. In light of these disadvantages, the therapeutic and societal benefits of rapid-acting insulin analogue formulations would be enhanced by the engineering of insulin analogues that retain rapid action at strengths between U-200 and U-1000. Additional benefits would accrue if the novel soluble insulin analogue exhibited weaker affinity for the Type 1 IGF receptor relative to wild-type human insulin; this provides an example of an off-target (or secondary target) effect. Still additional therapeutic and societal benefit would accrue if the concentrated insulin analogue formulation should exhibit reduced mitogenicity in assays developed to monitor insulin-stimulated proliferation of human cancer cell lines.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure.

Insulin is a small globular protein that plays a central role in metabolism in vertebrates. Insulin contains two chains, an A chain, containing 21 residues, and a B chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain. A variety of evidence indicates that it consists of an insulin-like core and disordered connecting peptide. Formation of three specific disulfide bridges (A6-A11, A7-B7, and A20-B19) is coupled to oxidative folding of a single-chain biosynthetic precursor, designated proinsulin, in the rough endoplasmic reticulum (ER). The sequence and structure of proinsulin are shown in schematic form in FIGS. 1A and 1B. Proinsulin is coverted to insulin in the trans-Golgi network en route to storage as zinc insulin hexamers in the glucose-regulated secretory granules within pancreatic beta-cells. The amino-acid sequences of the A- and B chains of human insulin and their disulfide pairing are shown in schematic form in FIG. 1C.

### SUMMARY OF THE INVENTION

The present invention was motivated by medical and societal needs to engineer a rapid-acting insulin analogue in a soluble formulation at neutral pH at strengths in the range U-200 through U-1000. Increasing the concentration of pradial insulin analogues that are known in the art favors their progressive self-association whereas the logic of their original design molecular design envisioned decreased self-association (Brange J 1, Ribel U, Hansen J F, Dodson G, Hansen M T, Havelund S, Melberg S G, Norris F, Norris K, Snel L, et al. (1988) Monomeric insulins obtained by protein engineering and their medical implications. Nature 333:679-82). This logic is illustrated in schematic form in FIG. 2. Traditional insulin formulations known in the art typically employ a predominance of zinc insulin hexamers at a nominal protein concentration, in monomer units, of 0.6 mM (lower on dilution), whose stable assembly protects the hormone from physical and chemical degradation. Concentrating wild-type insulin hexamers above this protein concentration leads to progressive hexamer-hexamer interactions; this further level of self-association is associated with delayed absorption of the injected insulin from a subcutaneous depot, leading in turn to prolonged pharmacokinetics and pharmacodynamics. Analogous prolongation of current prandial insulin analogue products (HUMALOG®, NOVOLOG® and APIDRA®) occurs on their concentration above ca. 2 mM (in monomer units).

A barrier to prandial formulations of increased strength in the range U-200 through U-1000 has therefore been posed by the complex self-association properties of wild-type insulin, which at neutral pH can form a concentration-dependent distribution of monomeric, dimeric, trimeric, tetrameric, hexameric, dodecameric, and higher-order species. To overcome this barrier, we have envisaged two novel routes toward the engineering of a rapid-acting insulin analogue formulation with increased strength. The first approach was to design ultra-stable insulin monomers and dimers refractory to higher-order self-assembly even at protein concentrations as high as 3-8 mM. In this approach the augmented intrinsic stability of the individual insulin analogue molecule would render its zinc-mediated or zinc-independent hexamer assembly unnecessary for a stable formulation, i.e., in accordance with guidelines of the U.S. Food & Drug Administration with respect to chemical degradation, polymerization and fibrillation. The second approach was to seek insulin analogues whose intrinsic biological activity, on a per molecule basis, would be greater than that of wild-type insulin. Enhanced intrinsic activity would enable protein solutions even at the conventional concentration of 0.6 mM (as in HUMALOG®, NOVOLOG® and APIDRA®) to exhibit a strength greater than U-100. Because key mechanisms of insulin degradation are more rapid in concentrated protein solutions than in dilute protein solutions, higher intrinsic potency would also enhance formulation stability relative to a corresponding insulin analogue formulation of the same strength but higher protein concentration. If conferred by the same molecular design, these two complementary approaches would reinforce each other to enable the simultaneous optimization of formulation strength, speed of action and stability.

The engineering of rapid-acting insulin analogues that exhibit enhanced biological potency on subcutaneous or intravenous injection in a mammal such that formulations of varying strength in the range U-100 through U-1000 may exhibit similar pharmacokinetic and pharmacodynamics properties poses three complementary challenges, each without prior solution. The present invention provides a single set of molecular designs that simultaneously enable each of these challenges to be surmounted. We address each in turn.

The first challenge is how to achieve higher potency in vivo. The problem is distinct from that posed by increased biochemical affinity of an insulin analogue for the insulin receptor, which represents merely the first step in a complex sequence of molecular and cellular events in vivo that ultimately leads to translocation of the Glut4 glucose transporter from an internal pool within target cells to the plasma membrane and the consequent reduction in blood-glucose concentration. It is well known in the art that human insulin and analogues with markedly different in vitro potencies (either with increased or decreased affinities for the insulin receptor) can be equipotent in terms of hypoglycaemic effect in a mammal (Vølund, A., Brange, J., Drejer, K., Jensen, I., Markussen, J., Ribel, U., Sørensen, A. R., and Schlichtkrull, J. (1991) In vitro and in vivo potency of insulin analogues designed for clinical use. Diabet. Med. 8:839-47). The challenge of designing insulin analogues with enhanced in vivo potency is further magnified by the lack of correlation between such potency and in vitro cellular assays of activity (e.g., tissue culture of mouse adipocytes or HEP-G2 hepatocytes; Vølund, A., et al. ibid.). Because of such lack of correlation and in particular because of the unenhanced potency of insulin analogues with increased affinity for the insulin receptor, it is not known in the art whether, even in principle, rapid-acting analogues of enhanced potency in vivo might exist. The first surprising aspect of the present invention is therefore that enhanced in vivo potency may indeed be achieved and that such enhancement occurs despite the net effect of concurrent molecular modifications that decrease the affinity of the insulin analogue for the insulin receptor (IR). Our design employs one or more acidic substitutions in association with a halogenic derivative of Phenylalanine at position B24; affinity is also reduced for Type 1 insulin-like growth factor (IGF) receptor (IGF-1R).

The second surprising aspect of the second invention is that such insulin analogues can at the same time be designed to exhibit impaired self-assembly—and therefore rapid action on subcutaneous assembly—and yet maintain sufficient stability with respect to chemical and physical degradation as to permit their safe and effective formulation as a practical insulin product. It comes as a further surprise that the above two properties of the insulin analogues may be accompanied by no elevation in their mitogenicity. The confluence of these three favorable features requires three concurrent modifications, any one of which alone would not provide such benefits and indeed would be expected to be deleterious to the safety or efficacy of an insulin analogue. The three modifications are (ii) substitution of Histidine by Aspartic Acid at position B10; (ii) introduction of one or more additional acidic modifications at positions A8, B29 and/or B29 or as a dipeptide C-terminal extension of the B-chain; and (iii) introduction of a halogen atom into the aromatic ring of Phenylalanine at position B24. Although not wishing to be constrained by theory, we envision that the enhanced potency in vivo of insulin analogues of the present invention reflects augmented cellular signaling at target tissues on receptor engagement.

We envisage that the products of the present invention will disproportionately benefits patients in Western societies with obesity, Type 2 diabetes mellitus and marked insulin resistance. Such clinical features pose a growing burden to under-represented minorities, including African-Americans, Hispanic-Americans and indigenous American tribes. Due to their enhanced biological activity per nanomole of protein, products of the present invention will also be useful in extending the reservoir life of insulin pumps and in enabling the miniaturization of such pumps.

It is, therefore, an aspect of the present invention to provide insulin analogues that provide rapid-acting pharmacokinetic and pharmacodynamics properties on subcutaneous injection. The analogues of the present invention contain Aspartic Acid at position B10 and Glutamic Acid at one or more of the following additional positions: A8, B28, and/or B29. The insulin analogues of the present invention also contain one or more halogen atoms (in place of hydrogen atoms) in the aromatic ring of Phenylalanine at position B24. It is an additional aspect of the present invention that absolute in vitro affinities of the insulin analogue for insulin receptor (isoforms IR-A and IR-B) are in the range of 5-100% of wild-type human insulin and so are unlikely to exhibit prolonged residence times in the hormone-receptor complex; while not wishing to be bound by theory, such prolonged residence times are believed to be associated with enhanced risk of carcinogenesis in mammals or more rapid growth of cancer cell lines in culture. It is yet an additional aspect of the present invention that absolute in vitro affinities of the insulin analogue for the Type 1 insulin-like growth factor receptor (IGF-1R) are in the range 10-100% relative to wild-type human insulin and so unlikely either to exhibit prolonged residence times in the hormone/IGF-1R complex or to mediate IGF-1R-related mitogenesis in excess of that mediated by wild-type human insulin.

The above combination of features is conferred by a novel combination of acidic amino-acid substitutions in concert with a halogenic modification of the aromatic ring of Phenylalanine at position B24. The structural environment of this aromatic ring relative to the surface of an insulin monomer is shown in FIG. 3. Although not wishing to be constrained by theory, we imagine that the halogenic modification at B24 perturbs the conserved B24-related binding pocket of the insulin receptor and IGF-1R receptor, mitigating the effect of AspB10 to prolong the residence time and enhance the affinity of the three homologous hormone-receptor complexes (IR-A, IR-B and IGF-1R). Also without wishing to be constrained by theory, we further imagine that non-additive effects of a negative charge at position B10 (as conferred by AspB10) in concert with one or more negative charges at A8, B28 and/or B29 (as conferred by acidic amino-acid substitutions at one or more of these sites) augments the strength of the insulin signal at the cellular level on hormone-receptor engagement. In this model, it is the latter augmentation that leads to enhanced in vivo potency of the insulin analogue (in relation to the regulation of blood-glucose concentration in a mammal with diabetes mellitus) when evaluated on a per-molecule basis.

In general, the present invention provides an insulin analogue containing Aspartic Acid at position B10, a halogen-substituted Phenylalanine derivative at position B24, and an acidic amino-acid substitution at one or more of the following three positions: A8, B28 and/or B29. The present invention thus pertains to a novel class of insulin analogues containing a combination of modifications that together provide the long-sought clinical advantages not conferred by any one of the constituent modifications. The analogues of the present invention may contain a variant B chain containing a dipeptide C-terminal extension (residue positions B31 and B32) in which at least one residue is an acidic residue as exemplified, but not restricted to, Glu-Glu, Glu-Ala, Ala-Glu, Glu-Asp, Ser-Asp and so forth. Alternatively, the analogues of the present invention may optionally contain truncation of the B-chain such that (i) residues B 1, B1-B2, or B1-B3 are absent and/or (ii) residue B30 is absent. In another version the analogues of the present invention may contain a Proline at position B29 in association with an acidic amino-acid substitution at position B28. In yet another version the analogues of the present invention may contain Tryptophan at position A13, Glutamic Acid at position A14, and/or Glycine at position A21.

### BRIEF DESCRIPTION OF THE SEVERAL VIEWS OF THE DRAWINGS

FIG. 1A is a schematic representation of the sequence of human proinsulin (SEQ ID NO:1) including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin (SEQ ID NOS:2 and 3) indicating the position of residues B27 and B30 in the B-chain.

FIG. 2 is a schematic representation of the pharmacokinetic principle underlying the design of prandial (rapid-acting) insulin analogues as known in the art. Whereas in a subcutaneous depot the insulin hexamer (upper left) is too large to efficiently penetrate into capillaries (bottom), more rapid uptake is mediated by the insulin dimer (center top) and insulin monomer (upper right). Prandial insulin products HUMALOG® and NOVOLOG® contain insulin analogues (insulin-lispro and aspart, respectively) with amino-acid substitutions at or near the dimerization surface of the zinc insulin hexamer such that its rate of disassembly is accelerated; prandial insulin product APIDRA® (insulin deglulisine) is formulated as zinc-free oligomers (as a coupled equlibria) that likewise exhibit rapid rates of diassasembly in the depot. The insulin analogues of the present invention provide isolated insulin monomers and weakly associating dimers whose augmented stability enables formulation in the absence of zinc-mediated assembly or zinc-free higher-order assembly. The hexamer at upper left depicts a T₃R^{f}₃ zinc hexamer in which the A chain is shown in green, and B chain in blue; three bound phenolic ligands are shown as CPK models in red. The dimer at center depicts a zinc-free T2 dimer in which the A chain is shown in red, and B chain in green (residues B1-B23 and B29-B30) or gray (B24-B28; anti-parallel beta-sheet); dimer-related inter-chain hydrogen bonds are shown in dotted line. A collection of crystallographic protomers (T, R and R^{f}) is shown at upper right wherein the A chain is shown in red, and B chain in blue (B1-B9) and green (B10-B30).

FIG. 3 is an electrostatic surface representation of the T-state insulin monomer in which the role of the side chain of PheB24 (shown in ajure as a stick model) is shown in relative to a crevice adjoining the hydrophobic core. The empty spaces around the edges of the B24 aromatic ring are sufficiently large to enable substitution of the ortho, meta or para protons by fluoro-aromatic, chloro-aromatic, or bromo-aromatic modifications. The protein surface is shaded red in regions of negative electrostatic potential, and blue in regions of positive electrostatic potential.

FIG. 4 provides an intravenous assay of the potency of insulin analogue (designated T-0337) in relation to KP-insulin. Diabetic Sprague-Dawley rats (time 0 blood glucose of 350-400 mg/dl) were injected in a tail vein with 10 μg of the indicated full-length insulin analog/300 g body weight or 9.4 μg of the des-B1-B3 insulin analog/300 g body weight. (A) Plot of blood-glucose concentration (vertical axis) as a function of time (horizontal axis). Symbols: ( [symbol] ; shaded square with border) diluent control (buffer only); ( [symbol] ; shaded square without border) insulin-lispro); ( [symbol] ) des-B1,B3-AspB10, ortho-fluoro-PheB24, GluB29-insulin (designated T-0337). (B) Alternative plot of the same data in relation to the percent change of the initial blood-glucose concentration (vertical axis). Symbols are as in panel A. The number of rats in each group was 9 (diluent control) or 5 (the insulin analogues); error bars, standard errors.

FIG. 5 provides an assay of the pharmacodynamics response of diabetic Sprague-Dawley rats to the subcutaneous injection of insulin analogues. Diabetic Sprague-Dawley rats (time 0 blood glucose of 350-400 mg/dl) were injected in a tail vein with 10 μg of the indicated full-length insulin analog/300 g body weight or 9.4 μg of the des-B1-B3 insulin analog/300 g body weight. (A) Plot of blood-glucose concentration (vertical axis) as a function of time (horizontal axis). Symbols: ( [symbol] ; shaded square with border) diluent control (i.e., buffer only); (▪) insulin-lispro); (◯) GlyA21 derivative of AspB10, ortho-fluoro-PheB24, GluB29-insulin (designated T-0347). (B) Alternative plot of the same data in relation to the percent change of the initial blood-glucose concentration (vertical axis). Symbols are as in panel A. The number of rats in each group was 12 (diluent control), 11 (insulin-lispro) or 4 (the present insulin analogue); error bars, standard errors.

FIG. 6 provides an intravenous assay of the potency of insulin analogues versus insulin-lispro. Diabetic Sprague-Dawley rats (time 0 blood glucose of 350-400 mg/dl) were injected in a tail vein with the indicated full-length insulin analog/300 g body weight. In each panel the dilutent control (buffer only) is indicated by a filled gray square with a border ( [symbol] ). (A, C and E) Plot of blood-glucose concentration (vertical axis) as a function of time (horizontal axis). (B, D and F) Respective plots of the same data in relation to the percent change of the initial blood-glucose concentration (vertical axis).

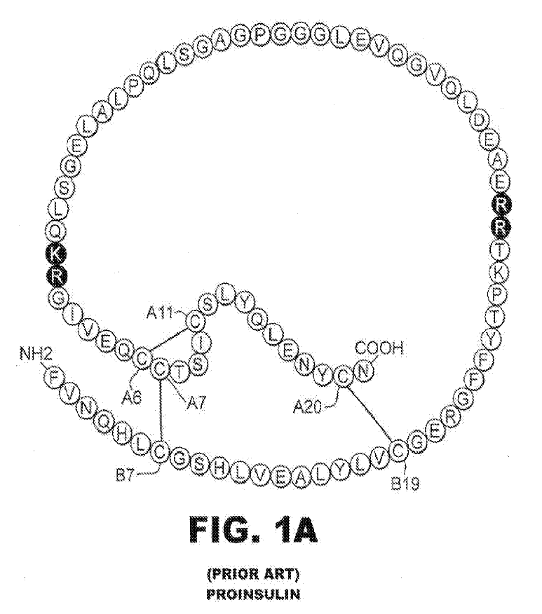

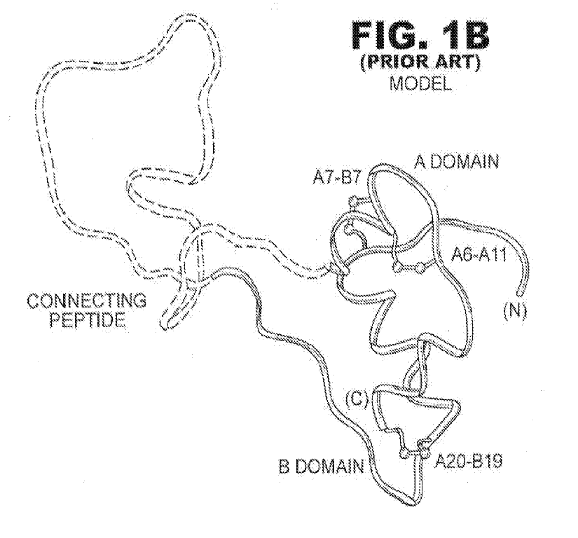

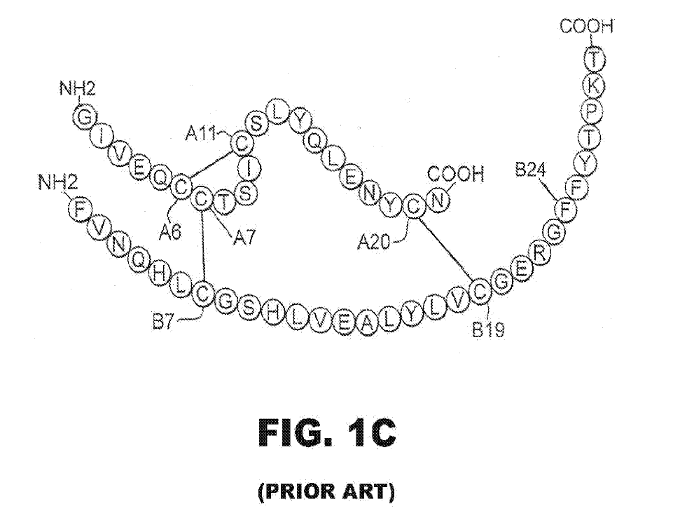

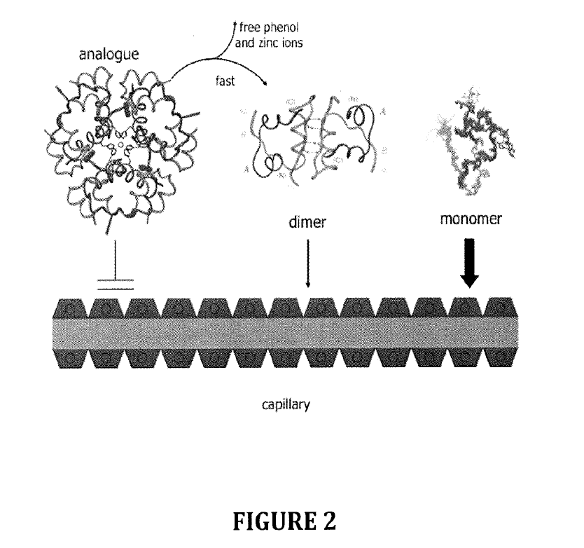

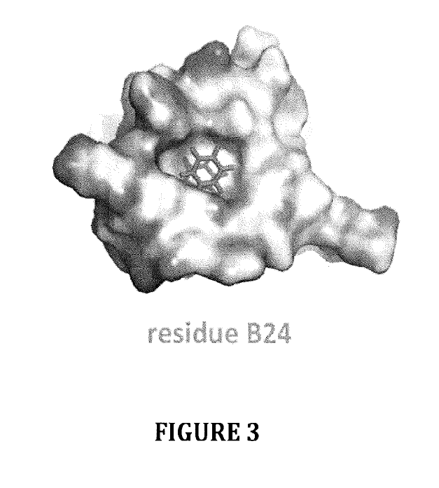

(A and B) Symbols: ( [symbol] ) AspB10, ortho-fluoro-PheB24, GluB29-insulin at 5 μg dose; ( [symbol] ) AspB10, ortho-fluoro-PheB24, GluB29-insulin at 10 μg dose; and ( [symbol] ; with no border) insulin-lispro at 10 μg dose. The number of rats in diluent group and in the insulin-lispro was in each case 9; for each dose of analogue T-0335, the number of rats was 4; error bars, standard errors. Analogue ortho-fluoro-PheB24, GluB29-insulin is also designated T-0335.

(C and D) Symbols: ( [symbol] GluA8 derivative of AspB10, ortho-fluoro-PheB24, GluB29-insulin at 10 μg dose; and z,21 ; no border) insulin-lispro at 10 μg dose. The number of rats in diluent group and in the insulin-lispro was in each case 9; for analogue T-0339, the number of rats was 4; error bars, standard errors. Analogue ortho-fluoro-PheB24, GluB29-insulin is also designated T-0339.

(E and F) Symbols: ( [symbol] ) GluA21, GluA8 derivative of AspB10, ortho-fluoro-PheB24, GluB29-insulin at 10 μg dose; and ( [symbol] ; no border) insulin-lispro at 10 μg dose. The number of rats in diluent group and in the insulin-lispro was in each case 9; for analogue T-0348, the number of rats was 4; error bars, standard errors. Analogue ortho-fluoro-PheB24, GluB29-insulin is also designated T-0348. Please note that the error bars for the present analogue (darker vertical line segments) in some cases smaller than the symbol and so are not visible in the figure.

FIG. 7 provides an assay of the pharmacodynamics response of diabetic Sprague-Dawley rats to the subcutaneous injection of insulin analogues. Diabetic rats (time 0 blood glucose of 400±20 mg/dl) were injected s.q. with 1 unit of the indicated insulin analog/300 g body weight. Diabetic Sprague-Dawley rats (time 0 blood glucose of 350-400 mg/dl) were injected in a tail vein with the indicated full-length insulin analog/300 g body weight. In each panel the dilutent control (buffer only) is indicated by a filled gray square with a border ( [symbol] ). (A, C and E) Plot of blood-glucose concentration (vertical axis) as a function of time (horizontal axis). (B, D and F) Respective plots of the same data in relation to the percent change of the initial blood-glucose concentration (vertical axis).

(A and B) Symbols: (▴) AspB10, ortho-fluoro-PheB24, GluB29-insulin at 20 μg dose; and (▪) insulin-lispro at 20 μg dose. The number of rats in diluent group was 12; in the insulin-lispro group the number of rats was 11; in the present analogue group the number of rats was 4; error bars, standard errors. Analogue ortho-fluoro-PheB24, GluB29-insulin is also designated T-0335.

(C and D) Symbols: (♦) AspB10, ortho-fluoro-PheB24, GluB29, GluB31, GluB32-insulin at 20 μg dose; and (▪) insulin-lispro at 20 μg dose. The number of rats in diluent group was 12; in the insulin-lispro group the number of rats was 11; in the present analogue group the number of rats was 4; error bars, standard errors. Analogue ortho-fluoro-PheB24, GluB29, GluB31, GluB32-insulin is also designated T-0336.

(E and F) Symbols: ( [symbol] )) GluA8 derivative of AspB10, ortho-fluoro-PheB24, GluB29-insulin at 10 μg dose; (X, black X) GluA8 derivative of AspB10, ortho-fluoro-PheB24, GluB29-insulin at 20 μg dose; ( [symbol] ; with no border) insulin-lispro at 10 μg dose; and (▪) insulin-lispro at 20 μg dose. The number of rats in diluent group was 12; in the insulin-lispro group at a dose of 20 μg the number of rats was 11; in the insulin-lispro group at a dose of 10 μg the number of rats was 7; in the present analogue group at each dose the number of rats was 11; error bars, standard errors. The GluA8 derivative of analogue ortho-fluoro-PheB24, GluB29-insulin is also designated T-0339.

FIG. 8 delineates the respective dose-response relationships of (B) a human insulin analogue (designated T-0339) containing acidic substitutions GluA8, AspB10, and GluB29 in concert with ortho-fluoro-Phenylalanine at position in relation to (A) control analogue KP-insulin. Data were in each case obtained from diabetic Sprague-Dawley rats following subcutaneous injection of the insulin analogues at the doses defined in the horizontal axis. The vertical axis provides the initial rate of decrease in the blood-glucose concentration during the first hour post injection. Respective IC50 values were estimated to be 5.4 μg (KP-insulin) and 2.9 μg (T-0339), consistent with a twofold enhancement of intrinsic potency in the analogue of the present invention.

FIG. 9 provides assays of thermodynamic stability as probed by chemical denaturation at 25° C. In these assays CD-detected ellipticity at a helix-sensitive wavelength (222 nm) is shown on the vertical axis as a function of the concentration of guanidine hydrochloride. Free energies of unfolding at zero denaturant concentration (ΔGᵤ) were inferred by application of a two-state model. (A) AspB10, 2F-PheB24, GluB29-human insulin (open circles; designated T-0335) versus KP-insulin (open circles; open squares). (B) AspB10, 2F-PheB24, GluB29, GluB31, GluB32-human insulin (open circles; designated T-0336) versus KP-insulin (open squares).

FIG. 10 also provides assays of thermodynamic stability as probed by chemical denaturation at 25° C. with CD-detected ellipticity at a helix-sensitive wavelength (222 nm) shown on the vertical axis as a function of the concentration of guanidine hydrochloride. Free energies of unfolding at zero denaturant concentration (ΔGᵤ) were inferred by application of a two-state model. (A) des-B1-B3 derivative of AspB10, 2F-PheB24, GluB29-human insulin (open circles; designated T-0337) versus KP-insulin (open squares). (B) GluA8 derivative of AspB10, 2F-PheB24, LysB28, ProB29-human insulin (open circles; designated T-0338) versus KP-insulin (open squares).

FIG. 11 provides assays of thermodynamic stability as probed by chemical denaturation at 25° C. as in FIGS. 9 and 10. (A) GluA8 derivative of AspB10, 2F-PheB24, GluB29-human insulin (open circles; designated T-0339) versus KP-insulin (open squares). (B) GluA8 derivative of AspB10, 2F-PheB24, GluB29, GluB31, GluB32-human insulin (open circles; designated T-0340) versus KP-insulin (open squares). 2F-PheB24 represents ortho-fluoro-Phenylalanine at position B24.

FIG. 12 provides assays of thermodynamic stability as probed by chemical denaturation at 25° C. as in FIGS. 9-11. (A) GlyA21 derivative of AspB10, 2F-PheB24, LysB28, ProB29-human insulin (open circles; designated T-0346) versus KP-insulin (open squares). (B) GlyA21 derivative of AspB10, 2F-PheB24, GluB29-human insulin (open circles; designated T-0347) versus KP-insulin (open squares).

FIG. 13 provides assays of thermodynamic stability as probed by chemical denaturation at 25° C. as in FIGS. 9-12. GluA8, GlyA21 derivative of AspB10, 2F-PheB24, GluB29-human insulin (open circles; designated T-0348) versus KP-insulin (open squares). In these assays CD-detected ellipticity at a helix-sensitive wavelength (222 nm) is shown on the vertical axis as a function of the concentration of guanidine hydrochloride. Free energies of unfolding at zero denaturant concentration (ΔGᵤ) were inferred by application of a two-state model. 2F-PheB24 represents ortho-fluoro-Phenylalanine at position B24.

FIG. 14 provides assays of mitogenicity in MCF-7 human breast cancer cell lines, enabling comparison of the analogues of the present invention to standards provided by wild-type human insulin, AspB10-human insulin, and insulin-like growth factor I (IGF-I; not shown).

### DETAILED DESCRIPTION OF THE INVENTION

The present invention is directed toward an insulin analogue that provides enhanced in vivo biological potency on a per-molecular basis, rapid action under a broad range of protein concentrations and formulation strengths (typically from U-100 to U-500 and optionally as high as U-1000), IR-A/IR-B receptor-binding affinities with absolute affinities in the range 5-100% relative to the affinities of wild-type human (the lower limit chosen to correspond to proinsulin), affinity for the IGF-1R no greater than that of wild-type human insulin, and increased thermodynamic stability in the absence of zinc ions relative to the baseline stability of wild-type human insulin in the absence of zinc ions.

It is a feature of the present invention that rapid absorption kinetics from a subcutaneous depot may be generated by an insulin analogue that is monomeric or dimeric—but not is a higher-order state of self-assembly—in a zinc-free solution at neutral pH at a protein concentration of 0.6-6.0 mM (as calculated in relation to the formal monomer concentration). Conventional prandial products, as known in the art, represent a continuum of possible coupled equilibria between states of self-assembly, including zinc-stabilized or zinc-ion-independent hexamers extended by potential hexamer-hexamer interactions. Molecular implementation of this strategy provides a novel class of insulin analogues that (i) are ultra-stable as a zinc-free monomer and dimer relative to wild-type human insulin and (ii) exhibit enhanced biological potency (as assessed by hormone-regulated reduction in blood-glucose concentration) on a per-molecular or per-nanomole basis. Although not wishing to be constrained by theory, the intrinsic stability of zinc-free insulin analogue monomers and dimers in the vial, pen or pump reservoir could enable stable formulation whereas the intrinsic potency of the analogues in the blood stream would provide the prandial glycemic control at formulation strengths U-200 through U-500 and optionally as high as U-1000; the augmented intrinsic stability would permit a given strength to be achieved at a lower protein concentration relative to current insulin analogue prandial formulations. It is a feature of the present invention that enhanced potency in relation to glycemic control is not associated with enhanced mitogenicity, a distinct signaling pathway that is undesirable from the perspective of cancer risk and cancer growth.

It is also envisioned that insulin analogues may be made with A- and B chain sequences derived from animal insulins, such as porcine, bovine, equine, and canine insulins, by way of non-limiting examples, so long as an Aspartic Acid is retained at position B10, a halogenated derivative of Phenylanaline is retained at position B24, and one or more acidic amino-acid substitutions are present at one or more of the sites provided by A8, B28 and/or B29. Such variant B chains derived from human insulin or animal insulins may optionally contain a C-terminal dipeptide extension (with respective residue positions designated B31 and B32) wherein at least one of these C-terminal extended residues is an acidic amino acid. In addition or in the alternative, the insulin analogue of the present invention may contain a deletion of residues B1-B3 or may be combined with a variant B chain lacking Proline at position B28 (e.g., AspB28 or GluB28 in combination with Lysine or Proline at position B29). At position A13 Leucine may optionally be substituted by Tryptophan, and at position A14 Tyrosine may optionally be substituted by Glutamic Acid.

It is further envisioned that the insulin analogues of the present invention may be derived from Lys-directed proteolysis of a precursor polypeptide in yeast biosynthesis in Pichia pastoris, Saccharomyces cerevisciae, or other yeast expression species or strains. Such strains may be engineered to insert halogen-modified Phenylalanine at position B24 by means of an engineered tRNA synthetase and orthogonal nonsense suppression. The B-domain of the insulin analogues of the present invention may optionally contain non-standard substitutions, such as D-amino-acids at positions B20 and/or B23 (intended to augment thermodynamic stability, receptor-binding affinity, and resistance to fibrillation). The halogenic modification at position B24 may be at the 2-ring position of Phe^{B24} (i.e., ortho-F-Phe^{B24}, ortho-Cl-Phe^{B24}, or ortho-Br-Phe^{B24}. Optionally, the analogues may contain iodo-substitutions within the aromatic ring of Tyr^{B16} and/or Tyr^{B26} (3-mono-iodo-Tyr or [3,5]-di-iodo-Tyr); intended to augment thermodynamic stability and receptor-binding activity). It is also envisioned that Thr^{B27}, Thr^{B30}, or one or more Serine residues in the C-domain may be modified, singly or in combination, by a monosaccaride adduct; examples are provided by O-linked N-acetyl-β-D-galactopyranoside (designated GalNAc-O^{β}-Ser or GalNAc-O^{β}-Thr), O-linked α-D-mannopyranoside (mannose-O^{β}-Ser or mannose-O^{β}-Thr), and/or α-D-glucopyrano side (glucose-O^{β}-Ser or glucose-O⁶² -Thr).

In various embodiments, alternative or additional mutations can be introduced into the insulin analogue described herein to affect the pharmacodynamics (e.g., onset or duration of action), receptor selectivity, glucose responsiveness, and/or stability (e.g., thermostability), and in some embodiments, the mutations render the insulin effective in concentrated form and/or suitable for delivery with pump systems.

The modifications described herein may be made in the context of any of a number of existing rapid acting insulin analogues such as Lispro insulin (Lys B28, Pro B29), insulin Aspart (Asp B28), and DKP-insulin. DKP insulin contains the substitutions Asp B10 (D), Lys B28 (K) and Pro B29 (P). In addition, or alternatively, the insulin analogue may contain one or more of the following modifications.

In some embodiments relating to a rapid-acting insulin analogue, the insulin analogue has an amino-acid substitution at position A8 (e.g., other than Glu). The A8 side chain is believed to project into solvent from the surface of the A-chain in both an insulin monomer and on its assembly into an insulin hexamer, thus enabling diverse side chains to be accommodated without steric clash. In the native structure of insulin this position is the C-terminal residue of the A1-A8 α-helix. Substitutions at A8 may enhance its C-Cap propensity (relative to the wild-type Thr) and hence augment the segmental stability of the A1-A8 α-helix. Diverse substitutions at A8, when introduced into rapid-acting insulin analogues containing B-chain substitutions known to the art, confer increased thermodynamic stability and increased resistance to fibrillation with substantial maintenance of the affinity for the insulin receptor relative to wild-type human insulin. Exemplary substitutions at A8 include tryptophan, methionine, lysine, or histidine. Other exemplary modifications in accordance with these embodiments are disclosed in U.S. Pat. No. 8,993,516, which is hereby incorporated by reference in its entirety.

In some embodiments, the insulin analogue provides for more rapid hexamer disassembly and hence accelerated absorption following subcutaneous injection. In some embodiments, as an alternative to halogenated Phe, the insulin analogue incorporates a non-standard amino-acid at position B24, such as Cyclohexanylalanine (Cha), which markedly enhances rapidity of hexamer disassembly, the rate-limiting step in insulin absorption in humans. This is achieved by substitution of an aromatic amino-acid side chain by a non-aromatic analogue, which is non-planar but of approximately similar size and shape to Phenylalanine, where the analogue then maintains at least a portion of biological activity of the corresponding insulin or insulin analogue containing the native aromatic side chain. See US 2014/0303076, which is hereby incorporated by reference. Further, additional substitutions of non-standard amino acids at position B29, for example, can provide for additional advantages. Exemplary non-standard amino acids include norleucine, aminobutryic acid, aminopropionic acid, ornithine, diaminobutyric acid, and diaminopropionic acid. See US 2014/0303076, which is hereby incorporated by reference in its entirety.

In still other embodiments, the insulin analogue exploits the dispensability of residues B1-B3 once disulfide pairing and protein folding have been achieved in the manufacturing process. Removal of residues B1-B3 can be accomplished through the action of trypsin on a precursor that contains Lys or Arg at position B3 in the place of the wild-type residue Asn B3. An example of such a precursor is the analog insulin glulisine, the active component of the product APIDRA® (Sanofi-Aventis). Analogs lacking residues PheB1-ValB2-AsnB3 thus contain a foreshortened B-chain (27 residues). The foreshortened B-chain confers resistance to fibrillation above room temperature while enabling native-like binding to the insulin receptor. Exemplary analogues of these embodiments are described in WO 2014/116753, which is hereby incorporated by reference in its entirety.

In some embodiments, the insulin analogue forms zinc-stabilized insulin hexamers of sufficient chemical stability and physical stability to enable their formulation at a range of protein concentrations and in a form that confers rapid absorption following subcutaneous injection. For example, the insulin analogue may have a set of three glutamic acid residues: Glu A8, Glu B31, and Glu B32, which may be used in combination with B-chain substitutions known in the art to cause accelerated disassembly of insulin hexamers or are associated with more rapid absorption of an insulin analogue following its subcutaneous injection relative to wild-type insulin in a similar formulation. For example, the insulin analogue may be modified by the incorporation of (a) Glutamic acid (Glu) at position A8, (b) a two-residue Glu B31-Glu B32 extension of the B-chain, and (c) optionally, a non-standard amino acid at position B24 (e.g., Cyclohexanylalanine or a halogenated derivative of the aromatic ring of Phenylalanine). See WO 2013/110069, which is hereby incorporated by reference in its entirety.

As discussed in more detail below, in some embodiments the insulin analogue addresses previous limitations for fast-acting insulin analogues, namely, that they are more susceptible to fibrillation than wild-type insulin. The insulin analogue may have an O-linked monosaccharide pyranoside adduct at B27 and/or B30 (e.g., mannopyranoside, N-acetyl-galactopyranoside, or glucopyranoside). These analogues exploit the natural occurrence of Threonine residues at positions B27 and B30 and the feasibility of trypsin-mediated semi-synthesis to attach synthetic peptides modified by carbohydrate adducts at these sites to the prefolded core of insulin (designated des,-octapeptide[B23-B30]-insulin; DOI). Exemplary insulin analogues in accordance with these embodiments are described in WO 2014/015078, which is hereby incorporated by reference.

In still other embodiments, rapid absorption of the insulin analogue into the blood stream is due at least in part to substitutions or modifications in or adjoining the Site-1-related surface of the B chain. Further, foreshortened duration of target cell signaling can be obtained by mutations or modifications of the Site-2-related surface of the A and/or B chain. Site-2-related substitutions are modifications at one or more of the following positions: B13, B17, A12, A13, and A17. See WO 2014/145593, which is hereby incorporated by reference in its entirety.

In some embodiments, the insulin analogue is a rapid acting insulin analogue comprising mono- or di- iodo-Tyr at B26 (e.g., 3-I-Tyr B26), which stabilizes the R6 hexamer, e.g., in a vial or delivery device. See U.S. Provisional Application No. 62/019,355, which is hereby incorporated by reference in its entirety.

In some embodiments, the insulin analogue displays glucose-responsive binding to the insulin receptor. Exemplary insulin analogues in accordance with these embodiments are disclosed in U.S. Provisional Application Nos. 62/132,704 and 62/133,251, which are hereby incorporated by reference. In some embodiments, such an analogue contains two essential elements. The first is a phenylboronic acid derivative (including a spacer element) at the α-amino group of Glycine at position A1 (Gly A1) or optionally at either the ε-amino group of D-Lysine as an amino-acid substitution well tolerated at position A1 (D-Lys A1) or the ε-amino group of L-Lysine as a substitution at position A4 (L-Lys A4). Phenylboronic acid groups bind to diols within saccharides. The spacer element may contain a linear acyl chain of 3-16 carbon atoms and optionally one or more nitrogen atoms at or near its terminus. The second element is a N-linked or O-linked monosaccharide, disaccharide, or oligosaccharide at one or more of the positions B27, B28, B29, B30, or as attached to a peptide extension of the B-chain containing one residue (B31) or two residues (B31-B32). Examples of O-linked saccharides are derivatives of Serine or Threonine; examples of N-linked saccharides are derivatives of Asparagine or Glutamine.

Examples of monosaccharides are glucose, mannose, and N-acetyl-galactose. The analogues may optionally contain an additional phenylboronic acid group (or halogenic derivative thereof) attached (together with a spacer element) to residue B1 as a mechanism intended to provide glucose-sensitive binding of the insulin analogue to surface lectins in the subcutaneous depot.

In some embodiments, the insulin analogue preferentially binds insulin receptor A (IR-A) relative to insulin receptor B (IR-B). Exemplary analogues and their beneficial properties are described in US 2011/0195896, which is hereby incorporated by reference in its entirety. For example, the analogue may be a single chain insulin where the insulin A chain and the insulin B chain are connected by a truncated linker compared to the linker of proinsulin. For example, the linker may be less than 15 amino acids long (e.g., 4 to 13 amino acids in length), and may have the sequence Gly-Pro-Arg-Arg in some embodiments.

In some embodiments, the insulin analogue is modified to decrease its relative affinity for the type I insulin-like growth factor receptor (IGFR), while substantially retaining or improving affinity for the insulin receptor (IR). Exemplary modifications in this respect are described in US 2012/0184488, which is hereby incorporated by reference in its entirety. For example, the insulin analogue may contain an amino acid addition at position A0 (that is, an addition at the amino terminal end of the A-chain) or amino-acid substitutions at positions A4, A8, or A21 or combinations thereof. In the native structure of insulin, residues A1-A8 comprise an α-helix. This segment is thought to contribute to the binding of insulin and insulin analogues to both IR and IGFR. In one example, the A0 extension is Arg, the A8 substitution is Arg, and the A21 substitution is Gly. In another example, the A0 extension is Arg, the A8 substitution is His, and the A21 substitution is Gly. In another example, the A4 substitution is His or Ala and the A8 substitution is His. In yet another example the A1 substitution is a D-amino acid and the A8 substitution is di-amino-butyric acid. Alternatively, as described in U.S. Provisional Application No. 62/105,713 (which is hereby incorporated by reference in its entirety), the analogue may contain Asp at B10 along with penta-Fluoro-Phe at B24, which may further be combined with Lys B3 and Glu B29.

Factors that accelerate or hinder fibrillation have been extensively investigated. Zinc-free insulin is susceptible to fibrillation under a broad range of conditions and is promoted by factors that impair native dimerization and higher order self-assembly. It is believed that the structure of active insulin is stabilized by axial zinc ions coordinated by the side chains of His B10. The insulin analogues sold under the trademark NOVOLOG® and HUMALOG® are associated with more rapid fibrillation and poorer physical stability. Fibrillation is a serious concern in the manufacture, storage and use of insulin and insulin analogues for diabetes treatment is enhanced with higher temperature, lower pH, for example.

In some embodiments, the insulin analogue comprises His A4 and His A8 together, and a histidine substitution at residue B1. It is believed that when the His B1 substitution is present, the side chain of the B1 His residue, in combination with the B5 histidine side chain, provides a potential B1-B5 bi-histidine Zn-binding site, which confers Zn-dependent protection from fibrillation. Similarly, it is believed that the His A4, His A8 substitutions also provide a potential bi-histidine Zn-binding site, which confers protection from fibrillation. Analogues in accordance with these embodiments are described in U.S. Pat. No. 8,343,914, which is hereby incorporated by reference.

In these or other embodiments, resistance to fibrillation is achieved, at least in part, by a halogenated phenylalanine at position B24, B25, and/or B26, which can be a chlorinated phenylalanine or a fluorinated phenylalanine. In various embodiments, the halogenated phenylalanine is ortho-monofluoro-phenylalanine, ortho-monobromo-phenylalanine, ortho-monochloro-phenylalanine or para-monochloro-phenylalanine. Exemplary analogues in accordance with these embodiments are disclosed in U.S. Pat. No. 8,921,313, which is hereby incorporated by reference in its entirety.

In still other embodiments, the insulin analogue exhibiting improvements in stability comprises a B-chain polypeptide containing at least one alteration selected from a methylated phenylalanine substitution at position B24 and an addition of two amino acids to the carboxyl end of the B-chain polypeptide. A first amino acid at position B31 is selected from glutamate and aspartate, and a second amino acid at position B32 is selected from glutamate, alanine and aspartate. The methylated phenylalanine may be ortho-monofluoro-phenylalanine, meta-monobromo-phenylalanine or para-monochloro-phenylalanine. These embodiments and others are described in U.S. Pat. No. 8,399,407, which is hereby incorporated by reference in its entirety. In some embodiments, the halogenated phenylalanine is penta-fluoro-phenylalanine, as described in US 2014/0128319, which is hereby incorporated by reference.

In some embodiments, resistance to fibrillation can be achieved at least in part through a single chain insulin comprising the structure described in U.S. Pat. No. 8,192,957, which is hereby incorporated by reference in its entirety. These embodiments combine amino-acid substitutions in the A- and B-chains of insulin with a linker peptide sequence such that the isoelectric point of the monomeric protein is similar to or less than that of wild-type human insulin, thereby preserving the solubility of the protein at neutral pH conditions. In some embodiments, the C peptide comprises an amino acid sequence selected from GGGPRR and GGPRR.

Furthermore, in view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Acidic amino acids are Aspartic acid (Asp or D) and Glutamic acid (Glu or E). Introduction of basic amino-acid substitutions (including Lysine (Lys or K), Arginine (Arg or R) and Histidine (His or H)) are not preferred in order to maintain the enhanced net negative charge of this class of analogues. Unless noted otherwise or wherever obvious from the context, the amino acids noted herein should be considered to be L-amino acids. Standard amino acids may also be substituted by non-standard amino acids belonging to the same chemical class.

The amino-acid sequence of human proinsulin is provided, for comparative purposes, as SEQ ID NO: 1.

SEQ ID NO: 1
(human proinsulin)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A chain of human insulin is provided as SEQ ID NO: 2.

SEQ ID NO: 2
(human A chain; residue positions A1-A21)
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B chain of human insulin is provided as SEQ ID NO: 3.

SEQ ID NO: 3
(human B chain; residue positions B1-B30)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of a modified insulin of the present invention is given in general form in SEQ ID NO: 4 wherein the six Cysteine residues are paired to provide three disulfide bridges as in wild-type human insulin.

SEQ ID NO: 4
(insulin analogue)
A chain
Gly-Ile-Val-Glu-Gln-Cys-Cys-Xaa₁-Ser-Ile-Cys-Ser-
Xaa₂- Xaa₃-Gln-Leu-Glu-Asn-Tyr-Cys-Xaa₄
SEQ ID NO: 5
(B chain)
Xaa₅-Xaa₆-Xaa₇-Gln-His-Leu-Cys-Gly-Ser-Asp-Leu-
Val-Glu-Ala-Leu-Tyr-Leu-Val-Cys-Gly-Glu-Arg-Gly-
Xaa₈-Phe-Tyr-Thr- Xaa₉- Xaa₁₀-Thr-Xaa₁₁-Xaa₁₂

Where Xaa₁ (position A8) may be Thr or Glu; where Xaa₂ may be Leu, Tyr or Trp; where Xaa₃ may be Tyr or Glu; where Xaa₄ may be Asn, Asp, Ala or Gly; where Xaa₅-Xaa₆-Xaa₇ may be Phe-Val-Asn as in wild-type human insulin or N-terminal deleted variants Val-Asn (des-B1), Asn (des-B1, B2) or omitted (des-B1-B3); where Xaa₈ is a derivative of Phenylalanine in which one or more hydrogen atoms in the aromatic ring are substituted by a halogen atom from the group fluorine (F), chlorine (C), or bromine (Br); where at least one of Xaa₉ or Xaa₁₀ is an acidic amino acid; and where optionally Xaa₁₁-Xaa₁₂ provides a C-terminal dipeptide extension of the B chain such that at least one residue is an acidic side chain.

The amino-acid sequences of insulin analogues of the present invention are in part given in SEQ ID NOS: 6-9 (containing intact B chains) and SEQ ID NOS:10-12 (containing N-terminally truncated B chains) wherein for brevity only the specific modifications relative to wild-type human insulin are provided (i.e., specific examples of sequence features Xaa₁, Xaa₂, Xaa₃, Xaa₄, Xaa₅, Xaa₆, Xaa₇, Xaa₈, Xaa₉, Xaa₁₀, Xaa₁₁ and Xaa₁₂. The molecules specified in SEQ ID NOS: 6-9 contain wild-type residues Phe-Val-Asn at respective B-chain positions B 1, B2, and B3 whereas the molecules specified in SEQ ID NOS:10-12 contain foreshortened B chains in which residues B1-B3 are absent. In each case residue B24 contains ortho-fluoro-Phenylalanine. The sequences provided in SEQ ID NO: 6 provide specific examples of insulin analogues in accordance with SEQ ID NO: 5 but these examples are not intended to circumscribe the combinatoric space of analogues defined by SEQ ID NO: 5. The sequence code provided pertains to an internal code of molecular designations.

| | | | other acidic/non- |
|---|---|---|---|
| ID/CODE | B10 | B24 halogen | acidic substitutions |
| SEQ ID | AspB10 | ortho-fluoro-PheB24 | GluB29 |
| NO: 4 + 5 | | | |
| T-0335 | | | |
| SEQ ID | AspB10 | ortho-fluoro-PheB24 | GluB29 GluB31 GluB32 |
| NO: 4 + 6 | | | |
| T-0336 | | | |
| SEQ ID | AspB10 | ortho-fluoro-PheB24 | GluA8 LysB28 ProB29 |
| NO: 4 + 8 | | | |
| T-0338 | | | |
| SEQ ID | AspB10 | ortho-fluoro-PheB24 | GluA8 GluB29 |
| NO: 4 + 9 | | | |
| T-0339 | | | |
| SEQ ID | AspB10 | ortho-fluoro-PheB24 | GlyA21 LysB28 ProB29 |
| NO: 4 + 8 | | | |
| T-0346 | | | |
| SEQ ID | AspB10 | ortho-fluoro-PheB24 | GlyA21 GluB29 |
| NO: 4 + 9 | | | |
| T-0347 | | | |
| SEQ ID | AspB10 | ortho-fluoro-PheB24 | GluA8 GlyA21 GluB29 |
| NO: 4 + 9 | | | |
| T-0348 | | | |

Analogues of the present invention may optionally contain N-terminal deletions of the B chain (des-B1, des-B1, B2 or des-B1-B3) as exemplified by, but not restricted to, SEQ ID NO: 10-12. These N-terminal residues are not required for receptor binding, but their presence in a biosynthetic single-chain precursor is thought to enhance the efficiency of native disulfide pairing in the endoplasmic reticulum and thus production yields.

| | | | other acidic/non- | B-chain deletion or |
|---|---|---|---|---|
| ID/CODE | B10 | B24 halogen | acidic substitutions | extension (ext.) |
| SEQ ID | AspB10 | ortho-fluoro-PheB24 | GluB29 | des-B1-B3 |
| NO: 4 + 10 | | | | no C-terminal ext. |
| T-0337 | | | | |
| SEQ ID | AspB10 | ortho-fluoro-PheB24 | GluB29 GluB31 GluB32 | des-B1-B3 |
| NO: 4 + 11 | | | | GluB31 GluB32 |
| T-0351 | | | | |
| SEQ ID | AspB10 | ortho-fluoro-PheB24 | GluA8 LysB28 ProB29 | des-B1-B3 |
| NO: 4 + 12 | | | | no C-terminal ext. |
| T-0338 | | | | |
| SEQ ID | AspB10 | ortho-fluoro-PheB24 | GluA8 GluB29 | des-B1-B3 |
| NO: 4 + 10 | | | | no C-terminal ext. |
| T-0339 | | | | |
| SEQ ID | AspB10 | ortho-fluoro-PheB24 | GlyA21 LysB28 ProB29 | des-B1-B3 |
| NO: 4 + 12 | | | | no C-terminal ext. |
| T-0355 | | | | |
| SEQ ID | AspB10 | ortho-fluoro-PheB24 | GlyA21 GluB29 | des-B1-B3 |
| NO: 4 + 10 | | | | no C-terminal ext. |
| T-0349 | | | | |
| SEQ ID | AspB10 | ortho-fluoro-PheB24 | GluA8 GlyA21 GluB29 | des-B1-B3 |
| NO: 4 + 10 | | | | no C-terminal ext. |
| T-0350 | | | | |

The following DNA sequences encode single-chain insulin analogues with codons optimized for usage patterns in Pichia pastoris. These single-chain insulin analogues provide biosynthetic intermediates for the production of the above two-chain insulin analogues. In each case the final codon (AAT) represents a stop codon.

The sense strand of a gene encoding a 53-residue single-chain insulin analogue with substitutions AspB10 and GluB30 and with C-domain Trp-Lys is given in SEQ ID NO: 13.

SEQ ID NO: 13
TTCGTCAATCAACACTTGTGTGGTAGTGACTTGGTCGAGGCATTGTACTT
GGTCTGTGGTGAGAGAGGATTCTTCTACACCCCAAAGGAGTGGAAGGGTA
TCGTTGAGCAATGTTGTACTTCCATCTGCTCATTGTACCAATTGGAGAAC
TACTGCAACTAA

The sense strand of a gene encoding a 53-residue single-chain insulin analogue with substitutions AspB10 and AlaB30 and with C-domain Ala-Lys is given in SEQ ID NO: 14.

SEQ ID NO: 14
TTCGTCAATCAACACTTGTGTGGTAGTGACTTGGTCGAGGCTTTGTACTT
GGTCTGTGGTGAGAGAGGATTCTTCTACACCCCTAAGGCTGCTAAGGGAA
TCGTTGAGCAATGCTGTACTTCCATCTGCTCATTGTACCAATTGGAGAAC
TACTGCAACTAA

The sense strand of a gene encoding a 53-residue single-chain insulin analogue with substitutions AspB10, GluA8 and GluB30 and with C-domain Trp-Lys is given in SEQ ID NO: 15.

SEQ ID NO: 15
TTCGTCAATCAACACTTGTGTGGTAGTGACTTGGTCGAGGCATTGTACTT
GGTCTGTGGTGAGAGAGGATTCTTCTACACCCCAAAGGAGTGGAAGGGTA
TCGTTGAGCAATGTTGTGAATCCATCTGCTCATTGTACCAATTGGAGAAC
TACTGCAACTAA

The sense strand of a gene encoding a 53-residue single-chain insulin analogue with substitution AspB10 and GluB30 and with C-domain Trp-Lys such that a non-standard amino acid may be inserted through nonsense suppression at codon position B24 (TAG) is given in SEQ ID NO: 16.

SEQ ID NO: 16
TTCGTCAATCAACACTTGTGTGGTAGTGACTTGGTCGAGGCATTGTACTT
GGTCTGTGGTGAGAGAGGATTCTTCTAGACCCCAAAGGAGTGGAAGGGTA
TCGTTGAGCAATGTTGTACTTCCATCTGCTCATTGTACCAATTGGAGAAC
TACTGCAACTAA

The sense strand of a gene encoding a 53-residue single-chain insulin analogue with substitution GluA8, AspB10 and GluB30 and with C-domain Trp-Lys such that a non-standard amino acid may be inserted through nonsense suppression at codon position B24 (TAG) is given in SEQ ID NO: 17.

SEQ ID NO: 17
TTCGTCAATCAACACTTGTGTGGTAGTGACTTGGTCGAGGCATTGTACTT
GGTCTGTGGTGAGAGAGGATTCTTCTAGACCCCAAAGGAGTGGAAGGGTA
TCGTTGAGCAATGTTGTGAATCCATCTGCTCATTGTACCAATTGGAGAAC
TACTGCAACTAA

The group of synthetic genes provided in SEQ ID NOS:18-22 provides a set of DNA sequences that optionally encode specific amino-acid substitutions at positions A13 and A14 in accordance with the amino-acid sequences specified above. It is known in the art that in the nuclear genes of yeasts, Leucine is encoded by DNA codons TTA, TTG, CTT, CTC, and CTG; that Tyrosine is encoded by DNA codons TAT and TAC; that Tryptophan is encoded by DNA codon TGG; and that Glutamic acid is encoded by DNA codons GAA and GAG.

SEQ ID NO: 18 provides the sense strand of a gene encoding a 53-residue single-chain insulin analogue with substitutions AspB10 and GluB30, with C-domain Trp-Lys such that the codon at position A13 (XXX₁) optionally encodes Leucine, Tyrosine or Tryptophan and such that the codon at position A14 (XXX₂) optionally encodes Tyrosine or Glutamic Acid.

SEQ ID NO: 18
TTCGTCAATCAACACTTGTGTGGTAGTGACTTGGTCGAGGCATTGTACTT
GGTCTGTGGTGAGAGAGGATTCTTCTACACCCCAAAGGAGTGGAAGGGTA
TCGTTGAGCAATGTTGTACTTCCATCTGCTCA-XXX₁-XXX₂-CAATTGG
AGAACTACTGCAACTAA

SEQ ID NO: 19 provides the sense strand of a gene encoding a 53-residue single-chain insulin analogue with substitutions AspB10 and AlaB30 and with C-domain Ala-Lys such that the codon at position A13 (XXX₁) optionally encodes Leucine, Tyrosine or Tryptophan and the codon at position A14 (XXX₂) optionally encodes Tyrosine or Glutamic Acid.

SEQ ID NO: 19
TTCGTCAATCAACACTTGTGTGGTAGTGACTTGGTCGAGGCTTTGTACTT
GGTCTGTGGTGAGAGAGGATTCTTCTACACCCCTAAGGCTGCTAAGGGAA
TCGTTGAGCAATGCTGTACTTCCATCTGCTCA-XXX₁-XXX₂-CAATTGG
AGAACTACTGCAACTAA

SEQ ID NO: 20 provides the sense strand of a gene encoding a 53-residue single-chain insulin analogue with substitutions AspB10, GluA8 and GluB30 and with C-domain Trp-Lys such that the codon at position A13 (XXX₁) optionally encodes Leucine, Tyrosine or Tryptophan and such that the codon at position A14 (XXX₂) optionally encodes Tyrosine or Glutamic Acid.

SEQ ID NO: 20
TTCGTCAATCAACACTTGTGTGGTAGTGACTTGGTCGAGGCATTGTACTT
GGTCTGTGGTGAGAGAGGATTCTTCTACACCCCAAAGGAGTGGAAGGGTA
TCGTTGAGCAATGTTGTGAATCCATCTGCTCA-XXX₁-XXX₂-CAATTGG
AGAACTACTGCAACTAA

SEQ ID NO: 21 provides the sense strand of a gene encoding a 53-residue single-chain insulin analogue with substitution AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan and such that the codon at position A14 (XXX₂) optionally encodes Tyrosine or Glutamic Acid.

SEQ ID NO: 21
TTCGTCAATCAACACTTGTGTGGTAGTGACTTGGTCGAGGCATTGTACTT
GGTCTGTGGTGAGAGAGGATTCTTCTAGACCCCAAAGGAGTGGAAGGGTA
TCGTTGAGCAATGTTGTACTTCCATCTGCTCA-XXX₁-XXX₂-CAATTGG
AGAACTACTGCAACTAA

SEQ ID NO: 22 provides the sense strand of a gene encoding a 53-residue single-chain insulin analogue with substitution GluA8, AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan and such the codon at position A14 (XXX₂) optionally encodes Tyrosine or Glutamic Acid.

SEQ ID NO: 22
TTCGTCAATCAACACTTGTGTGGTAGTGACTTGGTCGAGGCATTGTACTT
GGTCTGTGGTGAGAGAGGATTCTTCTAGACCCCAAAGGAGTGGAAGGGTA
TCGTTGAGCAATGTTGTGAATCCATCTGCTCA-XXX₁-XXX₂-CAATTGG
AGAACTACTGCAACTAA

The group of synthetic genes provided in SEQ ID NOS: 23-37 provides a set of DNA sequences that, in addition to the sequence features defined in SEQ ID NOS: 18-22, optionally encode a Lysine residue at one of the following three codon positions: B1 (SEQ ID NOS: 23-27), B2 (SEQ ID NOS: 28-32) or B3 (SEQ ID NOS: 33-37); such Lysine substitutions in a biosynthetic single-chain insulin precursor would enable production of insulin analogues of the present invention whose B chains contain N-terminal deletions des-B1, des-B1, B2, or des-B1-B3 in accordance with the amino-acid sequences specified above. These N-terminal truncations are respectively directed by substitution of Lysine at positions B1, B2 or B3 in the biosynthetic single-chain insulin precursor. It is known in the art that in nuclear genes of yeasts, Lysine is encoded by DNA codons AAA and AAG. As indicated above, it is also known in the art that in the nuclear genes of yeasts, Leucine is encoded by DNA codons TTA, TTG, CTT, CTC, and CTG; that Tyrosine is encoded by DNA codons TAT and TAC; that Tryptophan is encoded by DNA codon TGG; and that Glutamic acid is encoded by DNA codons GAA and GAG.

SEQ ID NOS: 23 provides the sense strand of a gene encoding a 53-residue single-chain insulin analogue with substitutions AspB10 and GluB30, with C-domain Trp-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 23
XXX₃GTCAATCAACACTTGTGTGGTAGTGACTTGGTCGAGGCATTGTACT
TGGTCTGTGGTGAGAGAGGATTCTTCTACACCCCAAAGGAGTGGAAGGGT
ATCGTTGAGCAATGTTGTACTTCCATCTGCTCA-XXX₁-XXX₂-CAATTG
GAGAACTACTGCAACTAA

SEQ ID NO: 24 provides the sense strand of a gene encoding a 53-residue single-chain insulin analogue with substitutions AspB10 and AlaB30 and with C-domain Ala-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 24
XXX₃GTCAATCAACACTTGTGTGGTAGTGACTTGGTCGAGGCTTTGTACT
TGGTCTGTGGTGAGAGAGGATTCTTCTACACCCCTAAGGCTGCTAAGGGA
ATCGTTGAGCAATGCTGTACTTCCATCTGCTCA-XXX₁-XXX₂-CAATTG
GAGAACTACTGCAACTAA

SEQ_ID NO:25 provides the sense strand of a gene encoding a 53-residue single-chain insulin analogue with substitutions AspB10, GluA8 and GluB30 and with C-domain Trp-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 25
XXX₃GTCAATCAACACTTGTGTGGTAGTGACTTGGTCGAGGCATTGTACT
TGGTCTGTGGTGAGAGAGGATTCTTCTACACCCCAAAGGAGTGGAAGGGT
ATCGTTGAGCAATGTTGTGAATCCATCTGCTCA-XXX₁-XXX₂-CAATTG
GAGAACTACTGCAACTAA

SEQ ID NO: 26 provides the sense strand of a gene encoding a 53-residue single-chain insulin analogue with substitution AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 26
XXX₃GTCAATCAACACTTGTGTGGTAGTGACTTGGTCGAGGCATTGTACT
TGGTCTGTGGTGAGAGAGGATTCTTCTAGACCCCAAAGGAGTGGAAGGGT
ATCGTTGAGCAATGTTGTACTTCCATCTGCTCA-XXX₁-XXX₂-CAATTG
GAGAACTACTGCAACTAA

SEQ ID NO: 27 provides the sense strand of a gene encoding a 53-residue single-chain insulin analogue with substitution GluA8, AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan, such the codon at position A14 (XXX₂) optionally encodes Tyrosine or Glutamic Acid, and such that XXX₃ encodes Lysine.

SEQ ID NO: 27
XXX₃GTCAATCAACACTTGTGTGGTAGTGACTTGGTCGAGGCATTGTACT
TGGTCTGTGGTGAGAGAGGATTCTTCTAGACCCCAAAGGAGTGGAAGGGT
ATCGTTGAGCAATGTTGTGAATCCATCTGCTCA-XXX₁-XXX₂-CAATTG
GAGAACTACTGCAACTAA

SEQ ID NO: 28 provides the sense strand of a gene encoding a 53-residue single-chain insulin analogue with substitutions AspB10 and GluB30, with C-domain Trp-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 28
TTCXXX₃AATCAACACTTGTGTGGTAGTGACTTGGTCGAGGCATTGTACT
TGGTCTGTGGTGAGAGAGGATTCTTCTACACCCCAAAGGAGTGGAAGGGT
ATCGTTGAGCAATGTTGTACTTCCATCTGCTCA-XXX₁-XXX₂-CAATTG
GAGAACTACTGCAACTAA

SEQ ID NO: 29 provides the sense strand of a gene encoding a 53-residue single-chain insulin analogue with substitutions AspB10 and AlaB30 and with C-domain Ala-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 29
TTCXXX₃AATCAACACTTGTGTGGTAGTGACTTGGTCGAGGCTTTGTACT
TGGTCTGTGGTGAGAGAGGATTCTTCTACACCCCTAAGGCTGCTAAGGGA
ATCGTTGAGCAATGCTGTACTTCCATCTGCTCA-XXX₁-XXX₂-CAATTG
GAGAACTACTGCAACTAA

SEQ ID NO: 30 provides the sense strand of a gene encoding a 53-residue single-chain insulin analogue with substitutions AspB10, GluA8 and GluB30 and with C-domain Trp-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 30
TTCXXX₃AATCAACACTTGTGTGGTAGTGACTTGGTCGAGGCATTGTACT
TGGTCTGTGGTGAGAGAGGATTCTTCTACACCCCAAAGGAGTGGAAGGGT
ATCGTTGAGCAATGTTGTGAATCCATCTGCTCA-XXX₁-XXX₂-CAATTG
GAGAACTACTGCAACTAA

SEQ ID NO: 31 provides the sense strand of a gene encoding a 53-residue single-chain insulin analogue with substitution AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 31
TTCXXX₃AATCAACACTTGTGTGGTAGTGACTTGGTCGAGGCATTGTACT
TGGTCTGTGGTGAGAGAGGATTCTTCTAGACCCCAAAGGAGTGGAAGGGT
ATCGTTGAGCAATGTTGTACTTCCATCTGCTCA-XXX₁-XXX₂-CAATTG
GAGAACTACTGCAACTAA

SEQ ID NO: 32 provides the sense strand of a gene encoding a 53-residue single-chain insulin analogue with substitution GluA8, AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan, such the codon at position A14 (XXX₂) optionally encodes Tyrosine or Glutamic Acid, and such that XXX₃ encodes Lysine.

SEQ ID NO: 32
TTCXXX₃AATCAACACTTGTGTGGTAGTGACTTGGTCGAGGCATTGTACT
TGGTCTGTGGTGAGAGAGGATTCTTCTAGACCCCAAAGGAGTGGAAGGGT
ATCGTTGAGCAATGTTGTGAATCCATCTGCTCA-XXX₁-XXX₂-CAATTG
GAGAACTACTGCAACTAA

SEQ ID NO: 33 provides the sense strand of a gene encoding a 53-residue single-chain insulin analogue with substitutions AspB10 and GluB30, with C-domain Trp-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 33
TTCGTCXXX₃CAACACTTGTGTGGTAGTGACTTGGTCGAGGCATTGTACT
TGGTCTGTGGTGAGAGAGGATTCTTCTACACCCCAAAGGAGTGGAAGGGT
ATCGTTGAGCAATGTTGTACTTCCATCTGCTCA-XXX₁-XXX₂-CAATTG
GAGAACTACTGCAACTAA

SEQ ID NO: 34 provides the sense strand of a gene encoding a 53-residue single-chain insulin analogue with substitutions AspB10 and AlaB30 and with C-domain Ala-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 34
TTCGTCXXX₃CAACACTTGTGTGGTAGTGACTTGGTCGAGGCTTTGTACT
TGGTCTGTGGTGAGAGAGGATTCTTCTACACCCCTAAGGCTGCTAAGGGA
ATCGTTGAGCAATGCTGTACTTCCATCTGCTCA-XXX₁-XXX₂-CAATTG
GAGAACTACTGCAACTAA

SEQ ID NO: 35 provides the sense strand of a gene encoding a 53-residue single-chain insulin analogue with substitutions AspB10, GluA8 and GluB30 and with C-domain Trp-Lys such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 35
TTCGTCXXX₃CAACACTTGTGTGGTAGTGACTTGGTCGAGGCATTGTACT
TGGTCTGTGGTGAGAGAGGATTCTTCTACACCCCAAAGGAGTGGAAGGGT
ATCGTTGAGCAATGTTGTGAATCCATCTGCTCA-XXX₁-XXX₂-CAATTG
GAGAACTACTGCAACTAA

SEQ ID NO: 36 provides the sense strand of a gene encoding a 53-residue single-chain insulin analogue with substitution AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan, such that the codon at position A14 (XXX₂) optionally encodes Tyrosine or Glutamic Acid, and such that XXX₃ encodes Lysine.

SEQ ID NO: 36
TTCGTCXXX₃CAACACTTGTGTGGTAGTGACTTGGTCGAGGCATTGTACT
TGGTCTGTGGTGAGAGAGGATTCTTCTAGACCCCAAAGGAGTGGAAGGGT
ATCGTTGAGCAATGTTGTACTTCCATCTGCTCA-XXX₁-XXX₂-CAATTG
GAGAACTACTGCAACTAA

SEQ ID NO: 37 provides the sense strand of a gene encoding a 53-residue single-chain insulin analogue with substitution GluA8, AspB10 and GluB30 and with C-domain Trp-Lys such that a non-standard amino acid may be inserted through nonsense suppression at codon position B24 (TAG), such that the codon at position A13 (XXX₁) optionally encodes Leucine, Tyrosine or Tryptophan, such the codon at position A14 (XXX₂) optionally encodes Tyrosine or Glutamic Acid, and such that XXX₃ encodes Lysine.

SEQ ID NO: 37
TTCGTCXXX₃CAACACTTGTGTGGTAGTGACTTGGTCGAGGCATTGTACT
TGGTCTGTGGTGAGAGAGGATTCTTCTAGACCCCAAAGGAGTGGAAGGGT
ATCGTTGAGCAATGTTGTGAATCCATCTGCTCA-XXX₁-XXX₂-CAATTG
GAGAACTACTGCAACTAA

Two single-chain insulin analogues of the present invention were prepared by biosynthesis of a precursor polypeptide in Pichia pastoris; this system secretes a folded protein containing native disulfide bridges with cleavage N-terminal extension peptide. Tryptic cleavage of this precursor protein yields a two-chain insulin fragment containing a truncated B chain beginning at residue PheB1 and ending at ArgB22 and a complete A chain. The precursor polypeptides are encoded by synthetic genes whose sequences are given in SEQ ID NOS: 19-28, which in each case contain the substitution AspB10 and may optionally contain the additional substitutions GluA8, TrpA13, TyrA13, and/or GluA14. Single-chain insulin precursors are also envisaged containing a nonsense codon at position B24 such that non-standard amino-acid substitutions may be inserted via an engineered orthogonal tRNA synthetase; such precursors would not be processed by trypsin but instead split by a lysine-specific endopeptidase.

The receptor-binding affinities of insulin analogues that exemplify the present invention were determined in relation to wild-type human insulin (Table 1). The assay employed the A isoform of the insulin receptor. Relative to human insulin and insulin-lispro (KP-insulin), the present insulin analogues retained relative affinities in the range 10-60%. The affinities of these analogues for the mitogenic Type 1 IGF-I receptor (IGF-1R) were similar to or weaker than that of wild-type human insulin (Table 2); one analogue containing AspB10 and ortho-fluoro-PheB24 but lacking a second acidic substitution in the B chain (designated T-0338) exhibited an affinity for the IGF-1R that was slightly stronger than that of wild-type insulin. The protocol for assay of receptor-binding activities was as follows. Microtiter strip plates (Nunc Maxisorb) were incubated overnight at 4° C. with AU5 IgG (100 μl/well of 40 mg/ml in phosphate-buffered saline). Binding data were analyzed by a two-site sequential model. Data were corrected for nonspecific binding (amount of radioactivity remaining membrane associated in the presence of 1 μM human insulin. In all assays the percentage of tracer bound in the absence of competing ligand was less than 15% to avoid ligand-depletion artifacts. Dissociation constants (K_{d}) were determined by fitting to a mathematic model as described by Whittaker and Whittaker (2005. J. Biol. Chem. 280: 20932-20936); the model employed non-linear regression with the assumption of heterologous competition (Wang, 1995, FEBS Lett. 360: 111-114).

TABLE 1
Affinities of Insulin Analogues for
the Insulin Receptor (Isoform B)

| | | standard | relative |
|---|---|---|---|
| Analog | K_{d} | error | affinity |
| Insulin | 0.16 | 0.003 | 100 |
| T-0335 (SEQ ID NO: 4 + 6) | 0.39 | 0.006 | 41 |
| T-0336 (SEQ ID NO: 4 + 7) | 0.91 | 0.015 | 17 |
| T-0338 (SEQ ID NO: 4 + 8) | 0.31 | 0.005 | 52 |
| T-0339 (SEQ ID NO: 4 + 9) | 0.37 | 0.005 | 43 |
| T-0346 (SEQ ID NO: 4 + 8) | 0.27 | 0.004 | 59 |
| T-0347 (SEQ ID NO: 4 + 9) | 0.38 | 0.006 | 42 |
| T-0348 (SEQ ID NO: 4 + 9) | 0.36 | 0.005 | 44 |

TABLE 2
Affinities of Insulin Analogues for
the Type 1 IGF Receptor (IGF-1R)

Dissociation Constant (nM)

| | | standard |
|---|---|---|
| Analog | K_{d} | error |
| T-0335 (SEQ ID NO: 4 + 6) | 31 | 6 |
| T-0336 (SEQ ID NO: 4 + 7) | 78 | 21 |
| T-0338 (SEQ ID NO: 4 + 8) | 7 | 1 |
| T-0339 (SEQ ID NO: 4 + 9) | 20 | 4 |
| T-0346 (SEQ ID NO: 4 + 8) | 10 | 2 |
| T-0347 (SEQ ID NO: 4 + 9) | 27 | 5 |
| T-0348 (SEQ ID NO: 4 + 9) | 24 | 5 |
| insulin | 11 | 2 |

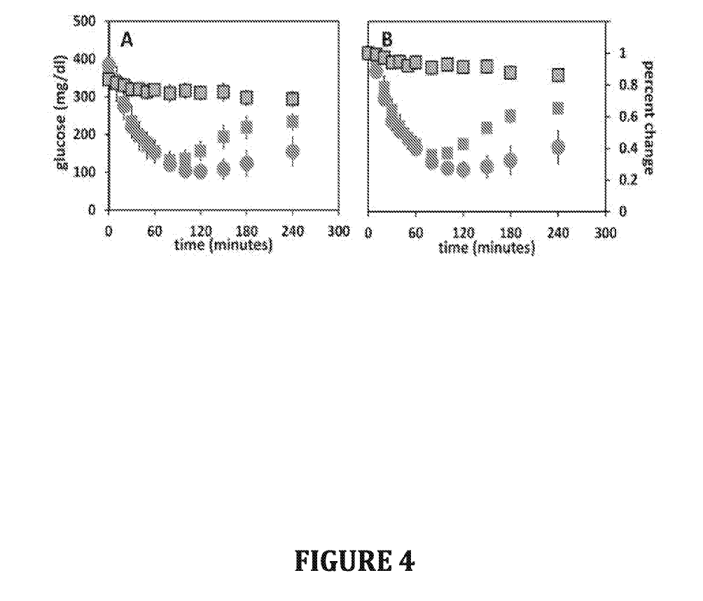

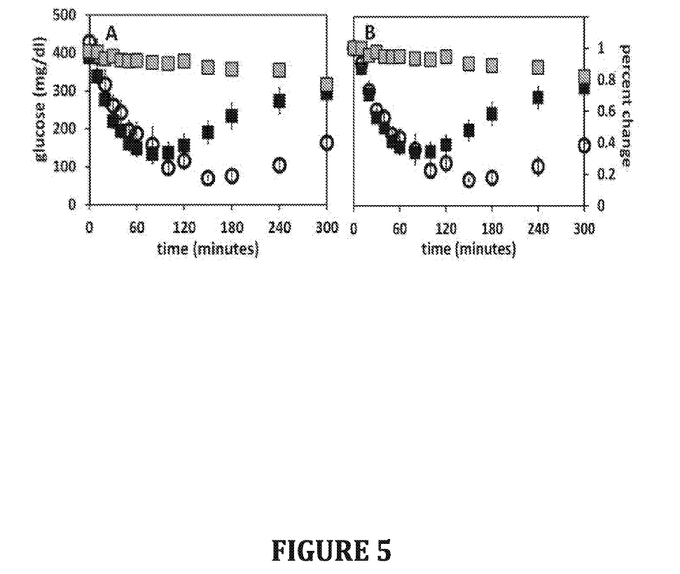

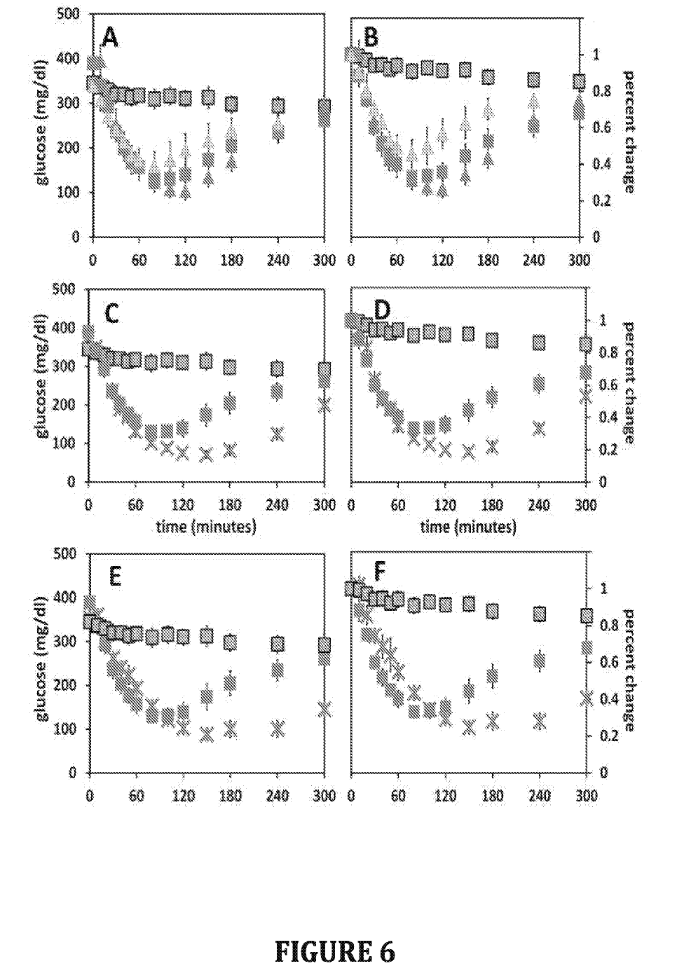

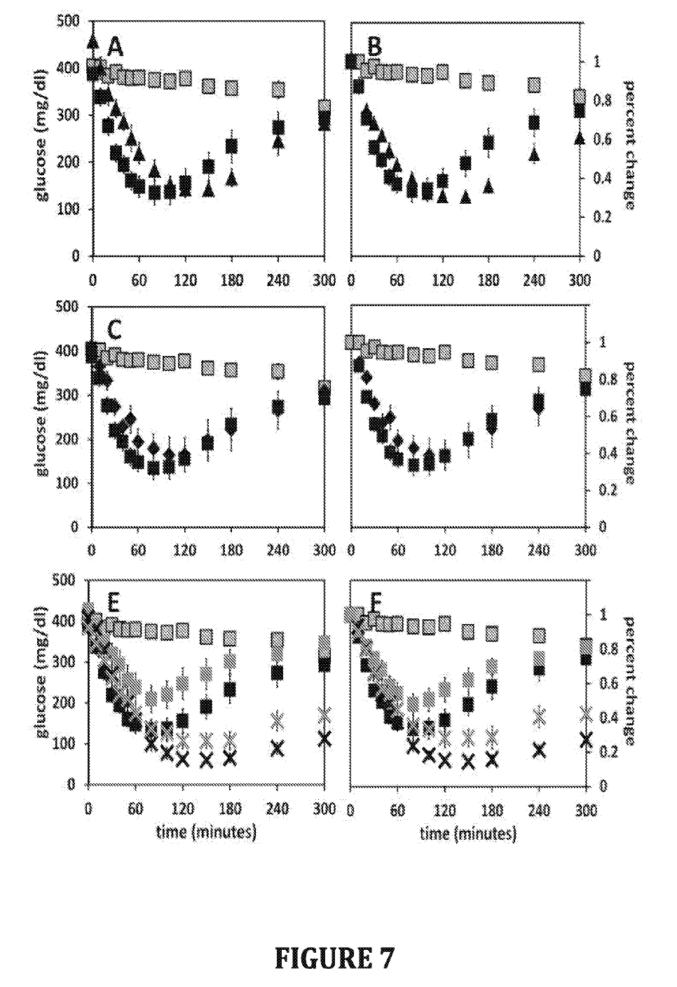

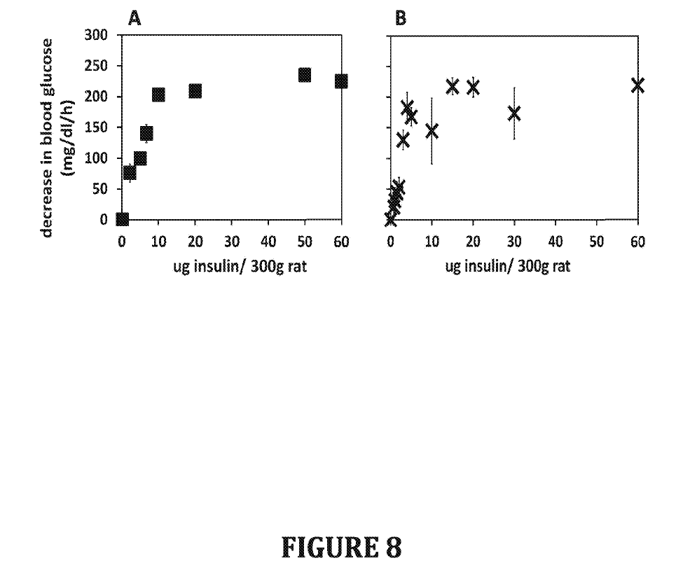

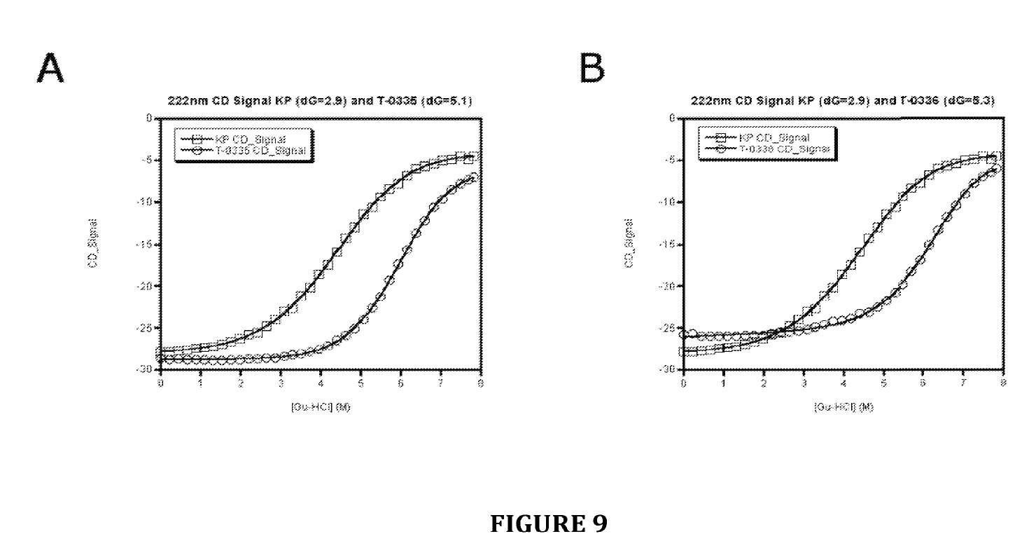

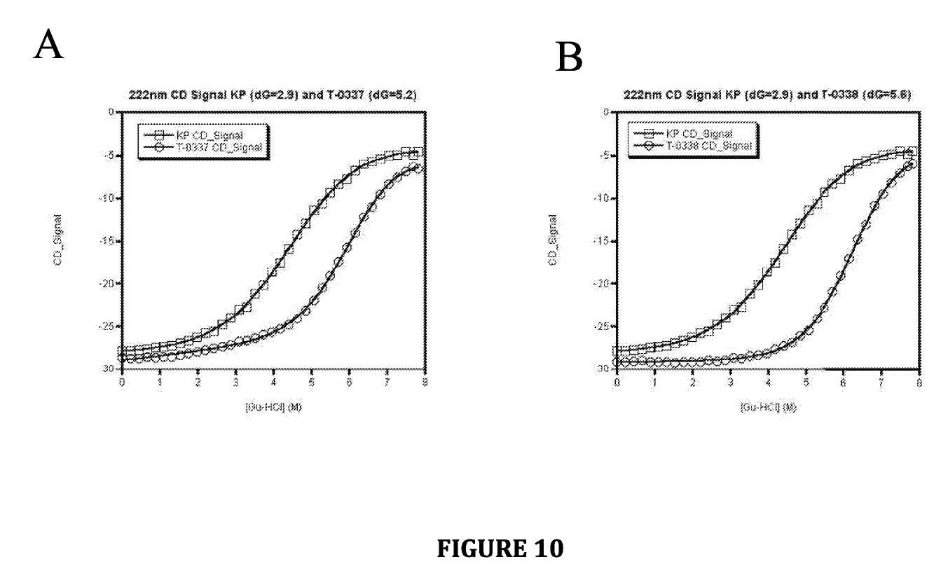

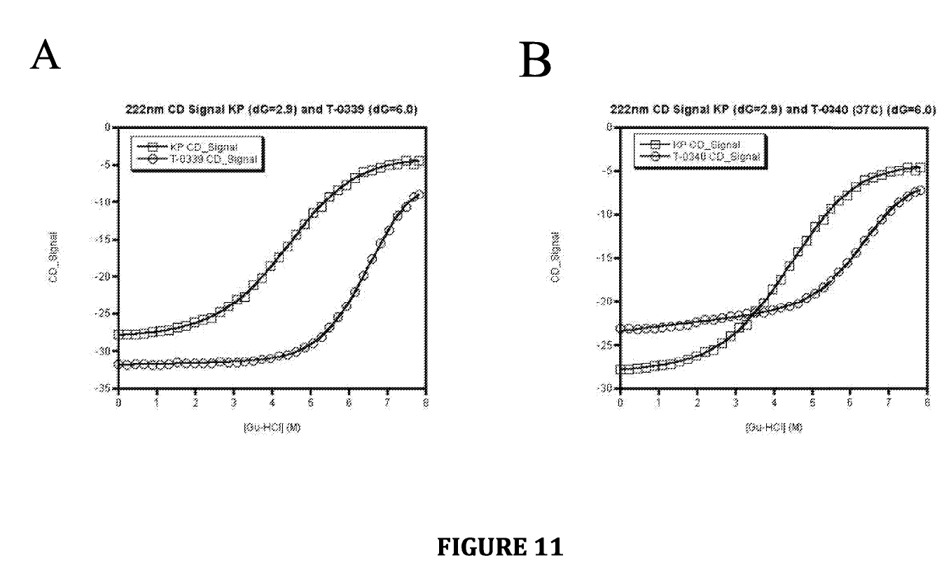

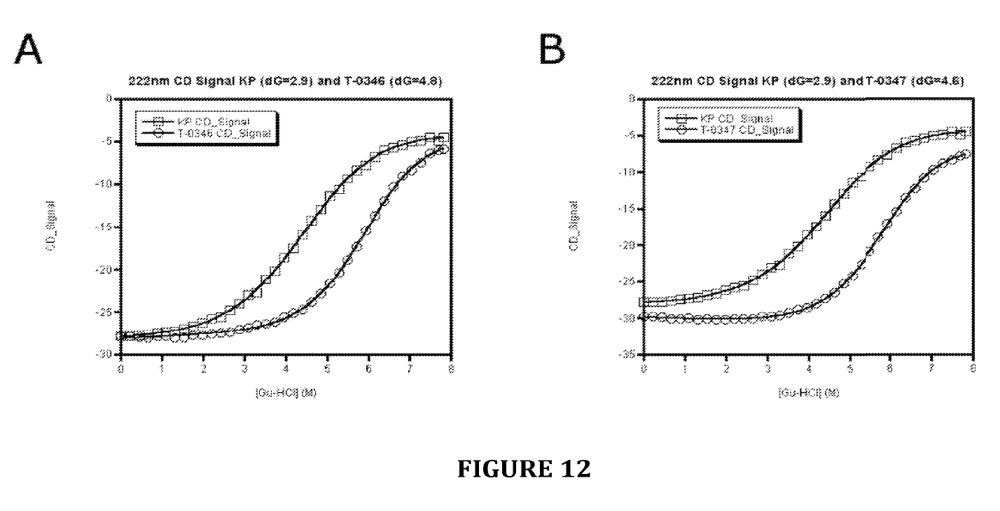

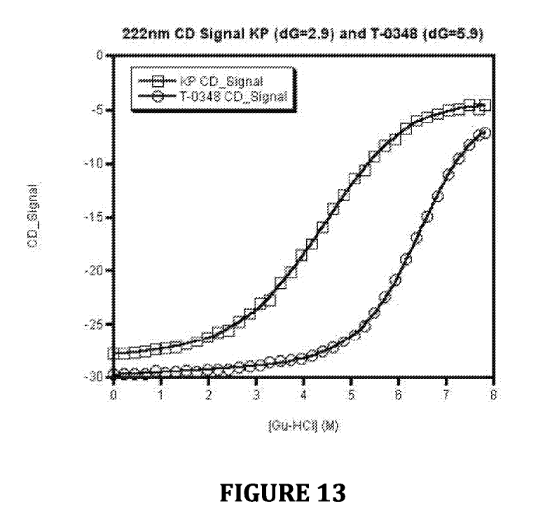

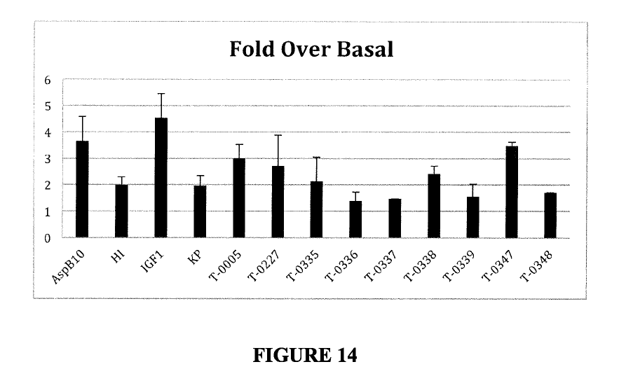

Biological activity and pharmacodynamics were tested in male Sprague-Dawley rats (ca. 300 g) rendered diabetic by streptozotocin. Intrinsic potency was evaluated by intravenous bolus injection of a specific dose of the insulin analogue into the tail vein of a rat at time t=0 min; representative data are provided in FIGS. 4 and 6. The pharmacodynamics responses of the rats to a subcutaneous injection at time t=0 min provides an integrated measure of both potency and bioavailability; the time course of insulin action in part reflects the rate of absorption of the insulin analogue from the subcutaneous depot. Representative data are provided in FIGS. 5 and 7. Initial rates of decline of the blood glucose concentration over the first hour following subcutaneous injection are shown in Tables 3A and 3B.

TABLE 3A
Initial Rate of Reduction of Blood-Glucose Concentration

| analogue | 10 μg dose^{a} | control^{b} |
|---|---|---|
| T-0338 | −239.8(±32.0) mg/dl/hr | −191.2(±20.8) mg/dl/hr |
| T-0346 | −194.3(±34.0) mg/dl/hr | −241.2(±21.4) mg/dl/hr |
| T-0348 | −175.3(±23.7) mg/dl/hr | −241.2(±21.4) mg/dl/hr |

^{a}Doses were adjusted, based on the actual mass of the individual rats, to correspond to 10 μg of insulin analogue per 300 gram body.
^{b}Control injections employed KP-insulin (insulin-lispro) at the same dose.

TABLE 3B
Initial Rate of Reduction of Blood-Glucose Concentration

| analogue | 20 μg dose^{a} | control^{b} |
|---|---|---|
| T-0335 | −230.5(±11.2) mg/dl/hr | −212.5(±22.1) mg/dl/hr |
| T-0336 | −212.1(±23.4) mg/dl/hr | −212.5(±22.1) mg/dl/hr |
| T-0337 | −315.2(±18.2) mg/dl/hr | −290.1(±17.6) mg/dl/hr |
| T-0339 | −272.4(±33.7) mg/dl/hr | −290.1(±17.6) mg/dl/hr |
| T-0340 | −292.9(±29.4) mg/dl/hr | −290.1(±17.6) mg/dl/hr |
| T-0347 | −254.7(±8.0) mg/dl/hr | −221.2(±24.4) mg/dl/hr |

^{a}Doses were adjusted, based on the actual mass of the individual rats, to correspond to 20 μg of insulin analogue per 300 gram body.
^{b}Control injections employed KP-insulin (insulin-lispro) at the same dose.

The analogues of the present invention exhibit greater potency on a per nanomole basis (relative to insulin-lispro) when injected by the intravenous or subcutaneous route into the diabetic Sprague-Dawley rats. Such enhancement was most evident at low doses of the analogue (i.e., at or below the IC₅₀ values) as exemplified by the data shown in FIG. 8. At doses of 10 μg per 300 gram body mass, or at greater doses, the analogues of the present invention effect similar reductions in blood-glucose concentration as does insulin-lispro (KP-insulin) within biological variability of the rats (from rat to rat and from week to week) as in apparent in Tables 3A and 3B.

The thermodynamic stabilities of the insulin analogues were probed by CD-monitored guanidine denaturation as described (Hua, Q. X., et al. J. Biol. Chem. 283, 14703-16 (2008)). The results indicate that these analogues are each more stable to chemical denaturation than are wild-type insulin or KP-insulin (respective free energies of unfolding (ΔGᵤ) at 25° C. 3.3±0.1 and 4.3±0.1 kcal/mole). The following estimates of ΔGᵤ at 25° C. were obtained by application of an analogous two-state model extrapolated to zero denaturant concentration: (analogue T-0335) 5.1±0.1 kcal/mole, (analogue T-0336) 5.3±0.1 kcal/mole, (analogue T-0338) 5.6±0.1 kcal/mole, (analogue T-0339) 6.0±0.1 kcal/mole, (analogue T-0346) 4.8±0.1 kcal/mole, (analogue T-0347) 4.6±0.1 kcal/mole, and (analogue T-0348) 5.9±0.1 kcal/mole. Such higher values of ΔGᵤ predict enhanced resistance of the present insulin analogues to chemical degradation under zinc-free conditions than would be observed in studies of wild-type insulin or KP-insulin under the same conditions. Representative data are provided in FIGS. 9 and 10. In each case higher concentrations of guanidine hydrochloride were required for 50% unfolding of the insulin analogues of the present invention than was required for the 50% unfolding of insulin-lispro (KP-insulin).

Assay for MCF-7 Colony Formation in Soft Agar. Assay for MCF-7 Colony Formation in Soft Agar. Single-cell suspensions were obtained by mixing a 0.25-ml suspension (2.25×105 cells) of MCF-7 cells in 2× growth medium/5% dialyzed fetal bovine serum (FBS)±50 nM of the insulin analogues with 0.25 ml of pre-warmed (42 oC) 0.6% agar suspension. This 0.3% suspension was poured onto a 0.5 ml layer of 0.6% agar in 24-well plates. The agar was overlaid with 1× growth medium/5% dialyzed FBS±50 nM of the insulin analogues and re-fed 3×/week for 12 days. Colonies (>60 μm) were counted on days 9 and 12. Representative data based on the colonies counted on day 9 are shown in FIG. 11.

A method for treating a patient with diabetes mellitus comprises administering a single-chain insulin analogue as described herein. It is another aspect of the present invention that the single-chain insulin analogues may be prepared either in yeast (Pichia pastoris) or subject to total chemical synthesis by native fragment ligation. The synthetic route of preparation is preferred in the case of non-standard modifications, such as D-amino-acid substitutions, halogen substitutions within the aromatic rings of Phe or Tyr, or O-linked modifications of Serine or Threonine by carbohydrates; however, it would be feasible to manufacture a subset of the single-chain analogues containing non-standard modifications by means of extended genetic-code technology or four-base codon technology (for review, see Hohsaka, T., & Sisido, M., 2012). It is yet another aspect of the present invention that use of non-standard amino-acid substitutions can augment the resistance of the single-chain insulin analogue to chemical degradation or to physical degradation. We further envision the analogues of the present invention providing a method for the treatment of diabetes mellitus or the metabolic syndrome. The route of delivery of the insulin analogue is by subcutaneous injection through the use of a syringe or pen device. An insulin analogue of the present invention may also contain other modifications, such as a halogen atom at positions B25 or B26. An insulin analogue of the present invention may also contain a foreshortened B-chain due to deletion of residues B1-B3.

A pharmaceutical composition may comprise such insulin analogues and which may optionally include zinc. Because the insulin analogues of the present invention do not form classical zinc-stabilized hexamers (and indeed do not require such assembly for stability), zinc ions may be included at varying zinc ion:protein ratios lower than are typically employed in formulations containing a predominance of insulin hexamers; such ratios may be in the range 0.01-0.10 moles of zinc ions per mole of insulin analogue. The pH of the formulation is in the range pH 7.0-8.0; a buffer (typically sodium phosphate or Tris-hydrochloride) may or may not be present. In such a formulation, the concentration of the insulin analogue would typically be between about 0.6-5.0 mM; concentrations up to 5 mM may be used in vial or pen; the more concentrated formulations (U-200 or higher) may be of particular benefit in patients with marked insulin resistance. Excipients may include glycerol, glycine, arginine, Tris, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

Based upon the foregoing disclosure, it should now be apparent that the two-chain insulin analogues provided will carry out the objects set forth hereinabove. Namely, these insulin analogues exhibit enhanced biological activity (as defined by the nanomoles of protein monomer required to lower the blood-glucose concentration in a mammal on subcutaneous or intravenous injection) such that rapid action is retain on concentration of the insulin analogue from 0.6 mM (as is typically employed in U-100 strength formulations known in the art) to 3.0 mM (as employed in the product Humulin® R U-500; Eli Lilly and Co.). It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.
Brange J 1, Ribel U, Hansen J F, Dodson G, Hansen M T, Havelund S, Melberg S G, Norris F, Norris K, Snel L, et al. (1988) Monomeric insulins obtained by protein engineering and their medical implications. Nature 333:679-82.
Barnes-Seeman, D., Beck, J., and Springer, C. (2014) Fluorinated compounds in medicinal chemistry: recent applications, synthetic advances and matched-pair analyses. Curr. Top. Med. Chem. 14:855-64.
Brange J, editor. (1987) Galenics of Insulin: The Physico-chemical and Pharmaceutical Aspects of Insulin and Insulin Preparations. Berlin: Springer Berlin Heidelberg.
Hohsaka, T., and Sisido, M. (2012) Incorporation of non-natural amino acids into proteins. Curr. Opin. Chem. Biol. 6, 809-15.
Kalra, S., Balhara, Y., Sahay, B., Ganapathy, B., and Das, A. (2013) Why is premixed insulin the preferred insulin? Novel answers to a decade-old question. J. Assoc. Physicians India 61, 9-11.
Liu, M., Hua, Q. X., Hu, S. Q., Jia, W., Yang, Y., Saith, S. E., Whittaker, J., Aryan, P., and Weiss, M. A. (2010) Deciphering the hidden informational content of protein sequences: foldability of proinsulin hinges on a flexible arm that is dispensable in the mature hormone. J. Biol. Chem. 285:30989-1001.
Voloshchuk, N., Zhu, A. Y., Snydacker D., and Montclare, J. K. (2009) Positional effects of monofluorinated phenylalanines on histone acetyltransferase stability and activity. Bioorg. Med. Chem. Lett. 19:5449-51.
Vølund, A., Brange, J., Drejer, K., Jensen, I., Markussen, J., Ribel, U., Sørensen, A. R., and Schlichtkrull, J. (1991) In vitro and in vivo potency of insulin analogues designed for clinical use. Diabet. Med. 8:839-47.
Wang, Z. X. (1995) An exact mathematical expression for describing competitive biding of two different ligands to a protein molecule FEBS Lett. 360: 111-114.
Whittaker, J., and Whittaker, L. (2005) Characterization of the functional insulin binding epitopes of the full-length insulin receptor. J. Biol. Chem. 280: 20932-20936.
Yang, Y., Petkova, A., Huang, K., Xu, B., Hua, Q. X., Ye, I. J., Chu, Y. C., Hu, S. Q., Phillips, N. B., Whittaker, J., Ismail-Beigi, F., Mackin, R. B., Katsoyannis, P. G., Tycko, R., and Weiss, M. A. (2010) An Achilles' heel in an amyloidogenic protein and its repair: insulin fibrillation and therapeutic design. J. Biol. Chem. 285:10806-21.
Yuvienco, C., More, H. T., Haghpanah, J. S., Tu, R. S., and Montclare, J. K. (2012) Modulating supramolecular assemblies and mechanical properties of engineered protein materials by fluorinated amino acids. Biomacromolecules 13:2273-8.

1. An insulin molecule having increased biological potency, the insulin comprising an A Chain peptide and a B-Chain peptide, wherein the insulin comprises:
an aspartic acid at the position corresponding to B10 of human insulin,
a glutamic acid at one or more of positions corresponding to A8, B28, and B29 of human insulin or a glycine substitution at the position corresponding to A21 of human insulin, and
a halogenated phenylalanine at the position corresponding to B24 of human insulin.

2. The insulin molecule of claim 1, wherein the insulin molecule has a dipeptide C-terminal extension of the B-chain polypeptide selected from: Glu-Glu, Glu-Ala, Ala-Glu, Glu-Asp, and Ser-Asp.

3. The insulin molecule of claim 1, comprising a truncation of B1, B1-B2, or B1-B3.

4. (canceled)

5. The insulin molecule of claim 1, additionally comprising a proline substitution at the position corresponding to B29 of human insulin.

6. (canceled)

7. The insulin molecule of claim 1, comprising
a glutamic acid at the position corresponding to B29 of human insulin.

8. The insulin molecule of claim 7, further comprising a truncation of residues B1-B3.

9. The insulin molecule of claim 7, further comprising glycine at A21.

10. The insulin molecule of claim 7, further comprising glutamic acid at A8.

11. The insulin molecule of claim 7, further comprising GluB31-GluB32 as a dipeptide extension of the B chain.

12. The insulin molecule of claim 1, comprising
a glutamic acid at the position corresponding to A8 of human insulin.

13. The insulin molecule of claim 12, further comprising: lysine at the position corresponding to B28 of human insulin and proline at the position corresponding to B29 of human insulin.

14. The insulin molecule of claim 12, further comprising glycine at the position corresponding to A21 of human insulin.

15. A pharmaceutical composition comprising the insulin molecule of claim 1, formulated at U-100 to U-1000.

16. (canceled)

17. The pharmaceutical composition of claim 15, formulated at U-500 to U-1000.

18. The pharmaceutical composition of claim 15, additionally comprising zinc at a ratio of 0.01 to 0.10 moles per mole of insulin.

19. The pharmaceutical composition of claim 18, formulated at a pH of 7 to 8, and optionally comprising a pH buffer.

20. (canceled)

21. A method for treating a patient with diabetes mellitus, comprising administering an insulin analogue to the patient, wherein the insulin analogue comprises:
an aspartic acid at the position corresponding to B10 of human insulin,
a glutamic acid at one or more of positions corresponding to A8, B28, and B29 of human insulin or a glycine substitution at the position corresponding to A21 of human insulin, and
a halogenated phenylalanine at the position corresponding to B24 of human insulin.

22. (canceled)

23. The method of claim 21, wherein the insulin analogue comprises a glutamic acid at the position corresponding to position B29 of human insulin.

24. The method of claim 21, wherein the insulin analogue comprises a glutamic acid at the position corresponding to position A8 of human insulin.

25. The pharmaceutical composition of claim 15, wherein the formulation is devoid of added zinc.


SEQUENCE LISTING (37 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 21 aa, Artificial Sequence
  synthetic construct
  MISC_FEATURE (8)..(8): X (position A8) may be Thr or Glu
  MISC_FEATURE (13)..(13): X may be Leu, Tyr or Trp
  MISC_FEATURE (14)..(14): X may be Tyr or Glu
  MISC_FEATURE (21)..(21): X may be Asn, Asp, Ala or Gly
GIVEQCCXSICSXXQLENYCX

SEQ ID NO: 5 - protein, 32 aa, Artificial Sequence
  synthetic construct
  MISC_FEATURE (1)..(3): X may be Phe Val Asn as in wild type human insulin or N terminal deleted variants Val Asn (des B1), Asn (des B1, B2) or omitted (des B1 B3)
  MISC_FEATURE (24)..(24): X is a derivative of Phenylalanine in which one or more hydrogen atoms in the aromatic ring are substituted by a halogen atom from the group fluorine (F), chlorine (C), or bromine (Br)
  MISC_FEATURE (28)..(29): X is an acidic amino acid
  MISC_FEATURE (31)..(32): X provides a C terminal dipeptide extension of the B chain such that at least one residue is an acidic side chain
XXXQHLCGSDLVEALYLVCGERGXFYTXXTXX

SEQ ID NO: 6 - protein, 30 aa, Artificial Sequence
  synthetic construct
  MISC_FEATURE (24)..(24): X is ortho fluoro Phenylalanine
FVNQHLCGSDLVEALYLVCGERGXFYTPET

SEQ ID NO: 7 - protein, 32 aa, Artificial Sequence
  synthetic construct
  MISC_FEATURE (24)..(24): X is ortho fluoro Phenylalanine
FVNQHLCGSDLVEALYLVCGERGXFYTPETEE

SEQ ID NO: 8 - protein, 30 aa, Artificial Sequence
  synthetic construct
  MISC_FEATURE (24)..(24): X is ortho fluoro Phenylalanine
FVNQHLCGSDLVEALYLVCGERGXFYTKPT

SEQ ID NO: 9 - protein, 30 aa, Artificial Sequence; identical to SEQ ID NO: 6
  synthetic construct
  MISC_FEATURE (24)..(24): X is ortho fluoro Phenylalanine

SEQ ID NO: 10 - protein, 27 aa, Artificial Sequence
  synthetic construct
  MISC_FEATURE (21)..(21): X is ortho fluoro Phenylalanine
QHLCGSDLVEALYLVCGERGXFYTPET

SEQ ID NO: 11 - protein, 29 aa, Artificial Sequence
  synthetic construct
  MISC_FEATURE (21)..(21): X is ortho fluoro Phenylalanine
QHLCGSDLVEALYLVCGERGXFYTPETEE

SEQ ID NO: 12 - protein, 27 aa, Artificial Sequence
  synthetic construct
  MISC_FEATURE (21)..(21): X is ortho fluoro Phenylalanine
QHLCGSDLVEALYLVCGERGXFYTKPT

SEQ ID NO: 13 - DNA, 162 bp, Artificial Sequence
  synthetic construct
ttcgtcaatcaacacttgtgtggtagtgacttggtcgaggcattgtacttggtctgtggtgagagaggattcttctacaccccaaaggagtggaagggtatcgttgagcaatgttgtacttccatctgctcattgtaccaattggagaactactgcaactaa

SEQ ID NO: 14 - DNA, 162 bp, Artificial Sequence
  synthetic construct
ttcgtcaatcaacacttgtgtggtagtgacttggtcgaggctttgtacttggtctgtggtgagagaggattcttctacacccctaaggctgctaagggaatcgttgagcaatgctgtacttccatctgctcattgtaccaattggagaactactgcaactaa

SEQ ID NO: 15 - DNA, 162 bp, Artificial Sequence
  synthetic construct
ttcgtcaatcaacacttgtgtggtagtgacttggtcgaggcattgtacttggtctgtggtgagagaggattcttctacaccccaaaggagtggaagggtatcgttgagcaatgttgtgaatccatctgctcattgtaccaattggagaactactgcaactaa

SEQ ID NO: 16 - DNA, 162 bp, Artificial Sequence
  synthetic construct
ttcgtcaatcaacacttgtgtggtagtgacttggtcgaggcattgtacttggtctgtggtgagagaggattcttctagaccccaaaggagtggaagggtatcgttgagcaatgttgtacttccatctgctcattgtaccaattggagaactactgcaactaa

SEQ ID NO: 17 - DNA, 162 bp, Artificial Sequence
  synthetic construct
ttcgtcaatcaacacttgtgtggtagtgacttggtcgaggcattgtacttggtctgtggtgagagaggattcttctagaccccaaaggagtggaagggtatcgttgagcaatgttgtgaatccatctgctcattgtaccaattggagaactactgcaactaa

SEQ ID NO: 18 - DNA, 162 bp, Artificial Sequence
  synthetic construct
  misc_feature (133)..(135): N is TTA, TTG, CTT, CTC, CTG, TAT, TAC, or TGG
  misc_feature (136)..(138): N is TAT, TAC, GAA or GAG
ttcgtcaatcaacacttgtgtggtagtgacttggtcgaggcattgtacttggtctgtggtgagagaggattcttctacaccccaaaggagtggaagggtatcgttgagcaatgttgtacttccatctgctcannnnnncaattggagaactactgcaactaa

SEQ ID NO: 19 - DNA, 162 bp, Artificial Sequence
  synthetic construct
  misc_feature (133)..(135): N is TTA, TTG, CTT, CTC, CTG, TAT, TAC, or TGG
  misc_feature (136)..(138): N is TAT, TAC, GAA or GAG
ttcgtcaatcaacacttgtgtggtagtgacttggtcgaggctttgtacttggtctgtggtgagagaggattcttctacacccctaaggctgctaagggaatcgttgagcaatgctgtacttccatctgctcannnnnncaattggagaactactgcaactaa

SEQ ID NO: 20 - DNA, 162 bp, Artificial Sequence
  synthetic construct
  misc_feature (133)..(135): N is TTA, TTG, CTT, CTC, CTG, TAT, TAC, or TGG
  misc_feature (136)..(138): N is TAT, TAC, GAA or GAG
ttcgtcaatcaacacttgtgtggtagtgacttggtcgaggcattgtacttggtctgtggtgagagaggattcttctacaccccaaaggagtggaagggtatcgttgagcaatgttgtgaatccatctgctcannnnnncaattggagaactactgcaactaa

SEQ ID NO: 21 - DNA, 162 bp, Artificial Sequence
  synthetic construct
  misc_feature (133)..(135): n is a, c, g, or t
  misc_feature (136)..(138): N is TAT, TAC, GAA or GAG
ttcgtcaatcaacacttgtgtggtagtgacttggtcgaggcattgtacttggtctgtggtgagagaggattcttctagaccccaaaggagtggaagggtatcgttgagcaatgttgtacttccatctgctcannnnnncaattggagaactactgcaactaa

SEQ ID NO: 22 - DNA, 162 bp, Artificial Sequence
  synthetic construct
  misc_feature (133)..(135): N is TTA, TTG, CTT, CTC, CTG, TAT, TAC, or TGG
  misc_feature (136)..(138): N is TAT, TAC, GAA or GAG
ttcgtcaatcaacacttgtgtggtagtgacttggtcgaggcattgtacttggtctgtggtgagagaggattcttctagaccccaaaggagtggaagggtatcgttgagcaatgttgtgaatccatctgctcannnnnncaattggagaactactgcaactaa

SEQ ID NO: 23 - DNA, 162 bp, Artificial Sequence
  synthetic construct
  misc_feature (1)..(3): N is AAA or AAG
  misc_feature (133)..(135): N is TTA, TTG, CTT, CTC, CTG, TAT, TAC, or TGG
  misc_feature (135)..(138): N is TAT, TAC, GAA or GAG
nnngtcaatcaacacttgtgtggtagtgacttggtcgaggcattgtacttggtctgtggtgagagaggattcttctacaccccaaaggagtggaagggtatcgttgagcaatgttgtacttccatctgctcannnnnncaattggagaactactgcaactaa

SEQ ID NO: 24 - DNA, 162 bp, Artificial Sequence
  synthetic construct
  misc_feature (1)..(3): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
nnngtcaatcaacacttgtgtggtagtgacttggtcgaggctttgtacttggtctgtggtgagagaggattcttctacacccctaaggctgctaagggaatcgttgagcaatgctgtacttccatctgctcannnnnncaattggagaactactgcaactaa

SEQ ID NO: 25 - DNA, 162 bp, Artificial Sequence
  synthetic construct
  misc_feature (1)..(3): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
nnngtcaatcaacacttgtgtggtagtgacttggtcgaggcattgtacttggtctgtggtgagagaggattcttctacaccccaaaggagtggaagggtatcgttgagcaatgttgtgaatccatctgctcannnnnncaattggagaactactgcaactaa

SEQ ID NO: 26 - DNA, 162 bp, Artificial Sequence
  synthetic construct
  misc_feature (1)..(3): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
nnngtcaatcaacacttgtgtggtagtgacttggtcgaggcattgtacttggtctgtggtgagagaggattcttctagaccccaaaggagtggaagggtatcgttgagcaatgttgtacttccatctgctcannnnnncaattggagaactactgcaactaa

SEQ ID NO: 27 - DNA, 162 bp, Artificial Sequence
  synthetic construct
  misc_feature (1)..(3): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
nnngtcaatcaacacttgtgtggtagtgacttggtcgaggcattgtacttggtctgtggtgagagaggattcttctagaccccaaaggagtggaagggtatcgttgagcaatgttgtgaatccatctgctcannnnnncaattggagaactactgcaactaa

SEQ ID NO: 28 - DNA, 162 bp, Artificial Sequence
  synthetic construct
  misc_feature (4)..(6): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcnnnaatcaacacttgtgtggtagtgacttggtcgaggcattgtacttggtctgtggtgagagaggattcttctacaccccaaaggagtggaagggtatcgttgagcaatgttgtacttccatctgctcannnnnncaattggagaactactgcaactaa

SEQ ID NO: 29 - DNA, 162 bp, Artificial Sequence
  synthetic construct
  misc_feature (4)..(6): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcnnnaatcaacacttgtgtggtagtgacttggtcgaggctttgtacttggtctgtggtgagagaggattcttctacacccctaaggctgctaagggaatcgttgagcaatgctgtacttccatctgctcannnnnncaattggagaactactgcaactaa

SEQ ID NO: 30 - DNA, 162 bp, Artificial Sequence
  synthetic construct
  misc_feature (4)..(6): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcnnnaatcaacacttgtgtggtagtgacttggtcgaggcattgtacttggtctgtggtgagagaggattcttctacaccccaaaggagtggaagggtatcgttgagcaatgttgtgaatccatctgctcannnnnncaattggagaactactgcaactaa

SEQ ID NO: 31 - DNA, 162 bp, Artificial Sequence
  synthetic construct
  misc_feature (4)..(6): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcnnnaatcaacacttgtgtggtagtgacttggtcgaggcattgtacttggtctgtggtgagagaggattcttctagaccccaaaggagtggaagggtatcgttgagcaatgttgtacttccatctgctcannnnnncaattggagaactactgcaactaa

SEQ ID NO: 32 - DNA, 162 bp, Artificial Sequence
  synthetic construct
  misc_feature (4)..(6): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcnnnaatcaacacttgtgtggtagtgacttggtcgaggcattgtacttggtctgtggtgagagaggattcttctagaccccaaaggagtggaagggtatcgttgagcaatgttgtgaatccatctgctcannnnnncaattggagaactactgcaactaa

SEQ ID NO: 33 - DNA, 162 bp, Artificial Sequence
  synthetic construct
  misc_feature (7)..(9): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcgtcnnncaacacttgtgtggtagtgacttggtcgaggcattgtacttggtctgtggtgagagaggattcttctacaccccaaaggagtggaagggtatcgttgagcaatgttgtacttccatctgctcannnnnncaattggagaactactgcaactaa

SEQ ID NO: 34 - DNA, 162 bp, Artificial Sequence
  synthetic construct
  misc_feature (7)..(9): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcgtcnnncaacacttgtgtggtagtgacttggtcgaggctttgtacttggtctgtggtgagagaggattcttctacacccctaaggctgctaagggaatcgttgagcaatgctgtacttccatctgctcannnnnncaattggagaactactgcaactaa

SEQ ID NO: 35 - DNA, 162 bp, Artificial Sequence
  synthetic construct
  misc_feature (7)..(9): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcgtcnnncaacacttgtgtggtagtgacttggtcgaggcattgtacttggtctgtggtgagagaggattcttctacaccccaaaggagtggaagggtatcgttgagcaatgttgtgaatccatctgctcannnnnncaattggagaactactgcaactaa

SEQ ID NO: 36 - DNA, 162 bp, Artificial Sequence
  synthetic construct
  misc_feature (7)..(9): N is AAA or AAG
  misc_feature (133)..(135): N isTTA, TTG, CTT, CTC, CTG, TAT, TAC, or TGG
  misc_feature (136)..(138): N isTAT, TAC, GAA or GAG
ttcgtcnnncaacacttgtgtggtagtgacttggtcgaggcattgtacttggtctgtggtgagagaggattcttctagaccccaaaggagtggaagggtatcgttgagcaatgttgtacttccatctgctcannnnnncaattggagaactactgcaactaa

SEQ ID NO: 37 - DNA, 162 bp, Artificial Sequence
  synthetic construct
  misc_feature (7)..(9): N is AAA or AAG
  misc_feature (133)..(135): N is TTA, TTG, CTT, CTC, CTG, TAT, TAC, or TGG
  misc_feature (136)..(138): N is TAT, TAC, GAA or GAG
ttcgtcnnncaacacttgtgtggtagtgacttggtcgaggcattgtacttggtctgtggtgagagaggattcttctagaccccaaaggagtggaagggtatcgttgagcaatgttgtgaatccatctgctcannnnnncaattggagaactactgcaactaa